(19) United States
(12) Patent Application Publication
(10) Pub. No.: US 2009/0202513 A1
(43) Pub. Date: Aug. 13, 2009

(54) RIBONUCLEASE AND THIAZOLIDINEDIONE COMPOUNDS AND THEIR USE IN METHODS TO TREAT CANCER

(75) Inventors: Maria E. Ramos-Nino, Milton, VT (US); Benjamin Littenberg, Burlington, VT (US)

Correspondence Address: WOLF GREENFIELD & SACKS, P.C., 600 ATLANTIC AVENUE, BOSTON, MA, 02210-2206, US

(73) Assignee: The University of Vermont and State Agriculture College, Burlington, VT (US)

(21) Appl. No.: 12/349,319

(22) Filed: Jan. 6, 2009

Related U.S. Application Data
(60) Provisional application No. 61/010,255, filed on Jan. 7, 2008.

(51) Int. Cl. A61K 38/46; A61P 35/00; C12N 9/22; C12Q 1/34

(52) U.S. Cl. 424/94.6; 435/199; 435/18

(57) ABSTRACT

The present invention relates to methods and kits for the treatment of cancer. A novel drug combination comprising a ribonuclease compound and a thiazolidinedione compound has been identified as producing a synergistic cytotoxicity effect in cancer cells. Methods and kits pertaining to the co-administration of these compounds are discussed herein.

### RELATED APPLICATIONS

This application claims the benefit under 35 U.S.C. § 119(e) of U.S. Provisional Application Ser. No. 61/010,255, entitled “Ribonuclease and Thiazolidinedione Compounds and their use in Methods to Treat Cancer,” filed on Jan. 7, 2008, which is herein incorporated by reference in its entirety.

### GOVERNMENT INTEREST

This invention was made with government support under government contract K01 CA104159 and K24 DK068380 awarded by the National Institutes of Health. The United States Government has rights in this invention.

### FIELD OF THE INVENTION

This invention pertains to the field of cancer treatment. The invention in some aspects includes methods of treating cancer that include administering a combination of a ribonuclease compound and a thiazolidinedione (TZD) compound.

### BACKGROUND OF INVENTION

Existing cancer chemotherapy is often either toxic, ineffective, or both. Cytotoxic ribonucleases (RNases) are minimally toxic to humans with moderate effects on cancer cells, and thus represent potential cancer therapeutic agents. Currently the amphibian-derived RNase, ranpirnase (U.S. Pat. No. 5,559,212), known as Onconase™, is in Phase III clinical trials for use in thoracic cancer, as an RNase pharmaceutical. The anticancer effect of Onconase™ has been documented in vitro (Halicka et al., Cell Prolif 2000, 33:407-417, Lee et al., J Surg Oncol 2000, 73:164-171, Darzynkiewicz et al., Cell Tissue Kinet 1988, 21:169-182, Rybak et al., J Natl Cancer Inst 1996, 88:747-753, Juan et al., Leukemia 1998, 8:1241-1248, Newton et al., Blood 2001, 97:528-535) and in vivo (Lee et al., J Surg Oncol 2000, 73:164-171, Rybak et al., J Natl Cancer Inst 1996, 88:747-753, Newton et al., Blood 2001, 97:528-535, Mikulski et al., J Natl Cancer Inst 1990, 82:151-153). RNase pharmaceuticals, however have shown limitations in their use due to inconsistent therapeutic efficacy (Ramos-Nino et al., Mol Cancer Ther 2005, 4:835-842), and renal toxicity (Vasandani et al., Cancer Chemother Pharmacol 1999, 44(2):164-9).

Thiazolidinedione (TZD) compounds have shown effectiveness in treating diabetes. In addition, although several studies have potentially linked TZD compounds with anti-tumorigenic effects (Han et al., Mol Cancer Ther 2006, 5:430-437; Yu et al., Hepatology 2006, 43:134-143), clinical trials to test the effectiveness of TZD compounds in cancer treatment have been described as disappointing (Grommes et al., Lancet Oncol 2004; 5:419-29, Burnstein et al., Breast Cancer Res Treat 2003; 79:391-7, Debrock et al., Br J Cancer 2003; 89:1409-12, Russo, Med Hypotheses 2007, 68:343-346). Some studies have suggested that long term use of these compounds may have pro-carcinogenic effects (Ramos-Nino et al., BMC Medicine 2007; 5:17), thus making them unlikely targets for anti-cancer therapeutic approaches.

### SUMMARY OF INVENTION

Aspects of the invention relate to treatment of cancer using a novel therapeutic combination: a ribonuclease such as ranpirnase, and a thiazolidinedione compound (TZD), such as rosiglitazone. Methods, compositions, and kits of the invention relate to co-administration of a ribonuclease compound and a TZD compound for decreasing cell viability, both in vitro and in vivo. Aspects of the invention further relate to the selection of patients for whom this therapeutic approach is appropriate.

Described herein are methods for treating a cancer in a subject through co-administration of an amount of a ribonuclease compound and an amount of a thiazolidinedione compound, wherein the compounds are co-administered in amounts therapeutically effective to treat the cancer in the subject. In some embodiments the ribonuclease compound is ranpirnase and the thiazolidinedione compound is rosiglitazone. In some embodiments the cancer is an adenocarcinoma such as lung, breast or prostate cancer. In some embodiments the cancer is mesothelioma. In certain embodiments the cancer is a solid tumor. In some embodiments the cancer exhibits high survivin expression. The subject may be a mammal such as a human.

According to one aspect of the invention, the ribonuclease compound and the thiazolidinedione compound are co-administered in one or more pharmaceutical compositions. The pharmaceutical compositions may further comprise at least one pharmaceutically acceptable carrier, diluent, excipient, or adjuvant. Methods of the invention may further comprise administering an additional chemotherapeutic agent to the subject. In some embodiments the subject who is treated by co-administration of a ribonuclease compound and a thiazolidinedione compound may have or be suspected of having cancer.

In some embodiments, co-administration of a ribonuclease compound and a thiazolidinedione compound comprises administering the ribonuclease compound at least every 1 day, 2 days, 3 days, 4 days, 5 days, 6 days, 1 week, 2 weeks, 3 weeks, 4 weeks, 5 weeks, 6 weeks, 7 weeks, 8 weeks, 2 months, 3 months, 4 months, 5 months, 6 months, 7 months, 8 months, 9 months, 10 months, 11 months, or 12 months. In some embodiments co-administration of a ribonuclease compound and a thiazolidinedione compound comprises administering the thiazolidinedione compound at least every 1 day, 2 days, 3 days, 4 days, 5 days, 6 days, 1 week, 2 weeks, 3 weeks, 4 weeks, 5 weeks, 6 weeks, 7 weeks, 8 weeks, 2 months, 3 months, 4 months, 5 months, 6 months, 7 months, 8 months, 9 months, 10 months, 11, or 12 months. In some embodiments the co-administration of the ribonuclease compound and the thiazolidinedione compound to the subject is repeated one or more times.

According to another aspect of the invention, methods for decreasing viability of a cancer cell are provided. Methods relate to contacting the cancer cell with a combination of a ribonuclease compound and a thiazolidinedione compound each in an amount effective such that the combination decreases the viability of the cancer cell. In some embodiments the ribonuclease compound is ranpirnase and the thiazolidinedione compound is rosiglitazone. The cancer cell, which may be a mammalian cell such as a human cell, can be contacted in vitro or in vivo. In some embodiments the cell is contacted simultaneously with the ribonuclease compound and the thiazolidinedione compound. In some embodiments the cancer is an adenocarcinoma such as lung, breast or prostate cancer. In certain embodiments the cancer is mesothelioma. In some embodiments the cancer is a solid tumor. In certain embodiments the cancer exhibits PI3K-dependent Fra-1 expression and/or high survivin expression.

According to another aspect of the invention, methods for assessing effectiveness of a combination of a ribonuclease compound and a thiazolidinedione compound for treatment of a cancer are provided. Methods involve contacting a cell of the cancer with a combination of a ribonuclease compound and a thiazolidinedione compound, and determining whether the contacted cell has reduced viability. Reduced viability indicates effectiveness of the combination of the ribonuclease compound and the thiazolidinedione compound for treatment of the cancer. In some embodiments the ribonuclease compound is ranpirnase and the thiazolidinedione compound is rosiglitazone. The cancer cell, which may be a mammalian cell such as a human cell, and which may be from a subject, can be contacted in vitro or in vivo. In some embodiments the cancer cell is in contact with the ribonuclease compound at the same time the cancer cell is in contact with the thiazolidinedione compound.

In some embodiments a combination of a ribonuclease compound and thiazolidinedione compound is selected as a treatment regimen for the subject if it is determined that contact with the combination of the ribonuclease compound and the thiazolidinedione compound reduces viability of the cancer cell. Determination of the viability of the contacted cancer cell may comprise comparing viability of the contacted cancer cell with viability of a control cancer cell. In certain embodiments the control cancer cell is a cancer cell not contacted with the combination of the ribonuclease compound and the thiazolidinedione compound. In some embodiments the cancer is an adenocarcinoma such as lung, breast or prostate cancer. In certain embodiments the cancer is mesothelioma. In some embodiments the cancer is a solid tumor. In certain embodiments the cancer is a cancer that exhibits PI3K-dependent Fra-1 expression and/or high survivin expression.

According to a further aspect of the invention, kits for determining a treatment regimen for a cancer are provided. In some embodiments kits of the invention comprise a ribonuclease compound, a thiazolidinedione compound, and instructions for use of the two compounds for determining whether the combination of the two compounds can be used as a treatment regimen for the cancer. In some embodiments the kit comprises a first container comprising the ribonuclease compound and a second container comprising the thiazolidinedione compound. In some embodiments the ribonuclease compound is ranpirnase and the thiazolidinedione compound is rosiglitazone. In some embodiments the cancer is an adenocarcinoma such as lung, breast or prostate cancer. In certain embodiments the cancer is mesothelioma. In some embodiments the cancer is a solid tumor. In certain embodiments the cancer exhibits PI3K-dependent Fra-1 expression and/or high survivin expression.

In another aspect of the invention, kits for treating a cancer are provided. In some embodiments kits of the invention comprise a ribonuclease compound, a thiazolidinedione compound, and instructions for use of a combination of the compounds for treating the cancer. In some embodiments a first container comprises the ribonuclease compound and a second container comprises the thiazolidinedione compound. In other embodiments a first container comprises the ribonuclease compound and the thiazolidinedione compound. In some embodiments the ribonuclease compound is ranpirnase and the thiazolidinedione compound is rosiglitazone. In some embodiments the cancer is an adenocarcinoma such as lung, breast or prostate cancer. In certain embodiments the cancer is mesothelioma. In some embodiments the cancer is a solid tumor. In certain embodiments the cancer exhibits PI3K-dependent Fra-1 expression and/or high survivin expression.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 presents graphs indicating that the combination of ranpirnase and rosiglitazone increased cytotoxicity after 48 h of treatment compared to ranpirnase or rosiglitazone alone in seven of twelve human cancer cell lines in vitro as determined by the methoxy-tetrazolium salt (MTS) viability assay test. FIG. 1A shows results of cell line T47D; FIG. 1B shows results of cell line MCF7. FIG. 1C shows results of cell line MDA-MD-231; FIG. 1D shows results of cell line LNCaP; FIG. 1E shows results of cell line DU-145; FIG. 1F shows results of cell line PC3; FIG. 1G shows results of cell line SK-OV3; FIG. 1H shows results of cell line CA-OV3; FIG. 1I shows results of cell line MP5; FIG. 1J shows results of cell line MP6; FIG. 1K shows results of cell line NCI-H292; FIG. 1L shows results of cell line H1792. C=medium only control; Onc 1=ranpirnase 1 μg/mL; Rosi20=rosiglitazone 20 μM; O+R=both. Bars represent mean fold changes±SEM of 8 samples per group. Experiments repeated twice. *=P≦0.05 in comparison to control; !=P≦0.05 in comparison to both single treatments.

FIG. 2 presents graphs indicating that the treatment of cancer cells with the combination of ranpirnase and rosiglitazone for 48 h increased the proportion of hypodiploid (subG₀/G₁) cells in five out of twelve human cancer cells lines, as determined by flow cytometry using Propidium Iodine. FIG. 2A shows results of cell line T47D; FIG. 2B shows results of cell line MCF7; FIG. 2C shows results of cell line MDA-MB-231; FIG. 2D shows results of cell line LNCaP; FIG. 2E shows results of cell line DU-145; FIG. 2F shows results of cell line PC3; FIG. 2G shows results of cell line SK-OV3; FIG. 2H shows results of cell line CA-OV3; FIG. 2I shows results of cell line MP5; FIG. 2J shows results of cell line MP6; FIG. 2K shows results of cell line NCI-H292; FIG. 2L shows results of cell line H1792. C=medium only control; Onc 1=ranpirnase 1 μg/mL; Rosi20=rosiglitazone 20 μM; O+R=both. Bars represent mean±SEM of 2 samples per group. Experiments repeated twice. *=P≦0.05 in comparison to control; !=P≦0.05 in comparison to both single treatments.

FIG. 3 presents graphs indicating cancer cells resistant to synergistic killing by the combination of ranpirnase and rosiglitazone that were tested for cell growth (by cell count) and apoptosis (by count of Annexin V-positive cells by flow cytometry) after 6 days of treatment. FIG. 3A-B show results of cell line PC3; FIGS. 3C-D show results of cell line H1792; FIGS. 3E-F show results of cell line MDA-MB-231. C=medium only control; Onc 1=ranpirnase 1 μg/mL; Rosi20=rosiglitazone 20 μM; O+R=both. Symbols represent mean±SEM of 2 samples per group. Experiments repeated twice. *=P≦0.05 in comparison to control; !=P≦0.05 in comparison to both single treatments.

FIG. 4 presents Western blots showing that synergistic down-regulation of Fra-1 by treatment with the combination of ranpirnase (1 μg/mL) and rosiglitazone (20 μM) for 48 h is cell dependent. MP5 with a PI3K-dependent Fra-1 expression, as determined by the use of the small molecule inhibitor LY294002 (20 μM), shows the most down-regulating effect. Down-regulation of Survivin is observed in both cell lines with FIG. 4A showing results with cell line MP5 and FIG. 4B showing results with cell line MP6. C=Medium only control. Mean±SEM of N=2 samples per group. Experiments repeated 2×. *=P≦0.05 in comparison to respective control; !=P≦0.05 in comparison to both single treatments (ranpirnase and rosiglitazone).

FIG. 5 presents Western blots showing synergistic down-regulation of Fra-1 by treatment with the combination of ranpirnase (1 μg/mL) and rosiglitazone (20 μM) for 48 h and a 3 hour recovery with complete media containing 0.5% FBS and 1 μM insulin in FIG. 5A shows results with cell line MDA-MB-231 and FIG. 5B shows results with cell line DU-145. The use of the small molecule inhibitor LY294002 (20 μM) shows a PI3K-dependent expression of Fra-1 and Survivin in both cell lines. Synergistic down-regulation of Survivin is observed in DU-145. C=Medium only control. Mean±SEM of N=2 samples per group. Experiments repeated 2×. *=P≦0.05 in comparison to respective control; !=P≦0.05 in comparison to both single treatments (ranpirnase and rosiglitazone).

FIG. 6 presents graphs indicating siRNA mediated knockdown of Fra-1, and Fra-1 immunofluorescence. Quantitative RTPCR of the cancer cell lines MP5, PC3, MDA-MB-231 and NCI-H292 showed that after transfection with RNAi constructs for Fra-1 (siFra-1) or scramble controls (siC), all cell lines demonstrate Fra-1 knock-down of more than 50% (FIG. 6A). Apoptosis measured as Annexin V-positive cells by flow cytometry (FIG. 6B) shows that Fra-1 knockdown increases apoptosis in three out the four cell lines tested. FIG. 6C shows results of immunofluorescence, using PCNA as a proliferation marker, and demonstrates that that Fra-1 co-localizes with PCNA in the nucleus and that cells undergoing apoptosis (marked by the arrow) have neither PCNA nor Fra-1 expression in the nucleus.

FIG. 7 presents a graph indicating that knock-down of Fra-1 in the NCI-H292 lung cancer cell line increases cell killing by ranpirnase (0.1, 1, or 10 μg/mL) in a concentration-dependent manner, but not by rosiglitazone (10 or 20 μM). The synergistic effect of ranpirnase (0.1 or 1 μg/mL) and rosiglitazone (10 or 20 μM) for 48 h is observed only in the scramble control (siC) transfected cell lines. The use of the PPARγ antagonist GW9662 (2 M) did not significantly modify the synergistic effect of ranpirnase and rosiglitazone. C Medium only control. Mean±SEM of N=8 samples per group. Experiments repeated 2×. *=P≦0.05 in comparison to respective control; !=P≦0.05 in comparison to both single treatments (ranpirnase and rosiglitazone).

FIG. 8 represents a kit of the invention. The kit (10) shown in FIG. 8 includes a set of containers for housing a compounds (12) or (14) such as a RNase or a TZD compound. As well as instructions (20). Additional components may also be included in the kit.

### DETAILED DESCRIPTION

Aspects of the invention relate to methods and compositions for the treatment of cancer. The invention relates, at least in part, to the surprising discovery that treatment of cancer cells with a combination of a ribonuclease such as ranpirnase, and a thiazolidinedione compound (TZD), such as rosiglitazone, produces a synergistic enhancement of cytotoxicity, relative to treatment of cancer cells with either compound alone. The combination of TZD compounds and RNases has not been previously proposed to treat or manage cancer or any other condition. Disclosed herein are methods for decreasing cell viability, both in vitro and in vivo, including the treatment of cancer, through co-administration of a TZD compound and an RNase. Methods for identifying cancer patients for whom this therapeutic approach would be advantageous are further disclosed, as are kits pertaining to methods of the invention.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Aspects of the invention relate to the use of ribonuclease proteins in mediating cytotoxicity and in the treatment of cancer. Ribonuclease proteins (also referred to herein as RNases or RNase compounds) refer to enzymes that catalyze the hydrolysis of ribonucleic acid (RNA), and mediate intracellular degradation of RNA. RNases, including endoribonucleases and exoribonucleases, fall into multiple subclasses of the enzyme class EC 3.1 (Ramos-Nino, Drugs of the Future 2007, 32:517-526). Both endogenous and exogenous RNases can be used to mediate cellular toxicity. The use of RNases in therapeutics is discussed further in US Patent Publication No. 20050261232.

One of the earliest RNases for which cytotoxicity was investigated was bovine pancreatic RNase A (Ledoux, et al., Experientia 1954 10(12):500-1, Ledoux, Nature 1955, 175(4449):258-9; Ledoux, Nature 1955, 176(4470):36-7). Subsequently, a higher level of cytotoxicity than that exhibited by RNase A, was shown with two other classes of RNases: BS-RNases, isolated from bovine seminal vesicles (Matousek, Comp Biochem Physiol C Toxicol Pharmacol 2001, 129(3):175-91, Hosokawa et al., J Biochem (Tokyo) 1971, 69(4):683-97, Dostal et al., J Reprod Fertil 1973, 33(2):263-74, Matousek et al., Comp Biochem Physiol A 1973, 46(2):241-8, D'Alessio et al., FEBS Lett 1972, 27(2):285-8, Matousek, Experientia 1973, 29(7):858-9), and RNases derived from the eggs and embryos of frogs (Sakakibara et al., Biochim Biophys Acta 1976, 444(2):386-95, Nitta et al., Cancer Res 1994, 54(4):928-34, Ardelt et al., J Biol Chem 1991, 266(1):245-51, Darzynkiewicz et al., Cell Tissue Kinet 1988, 21:169-82). Ranpirnase refers to an RNase extracted from Rana pipiens, the Northern leopard frog, and has the registered trademark name ONCONASE™ (U.S. Pat. No. 5,559,212).

It should be appreciated that RNases from multiple sources are compatible with methods of the claimed invention. In some embodiments, the RNase is derived from frogs, such as the genus Rana, including Rana pipiens. In certain embodiments, the RNase is ranpirnase. In other embodiments, the RNase is a mammalian RNase such as a bovine RNase. In some embodiments, the RNase is a human RNase. Human RNases can be modified such that their activities will not be inhibited in human cells, an approach that is discussed further in U.S. Pat. Nos. 5,389,537, 6,280,991, 5,840,296, and US Patent Publication No. 20070003537. In some embodiments of the invention, the RNase is purified from an animal or human tissue, while in other embodiments the RNase is expressed and purified as a recombinant protein in bacteria, discussed further in US Patent Publication Nos. 20030027311 and 20050014161. RNases consistent with the invention include variants, such as RNases in which the sequence has been modified from its naturally occurring sequence. In some embodiments, the sequence of the RNase is modified to target the RNase to a cancer cell. Targeting of RNases is discussed further in U.S. Pat. No. 6,175,003.

### Thiazolidinedione Compounds

Aspects of the invention relate to combining RNases with thiazolidinedione (TZD) compounds to mediate cytotoxicity. A “TZD compound” as used herein refers to a synthetic ligand and agonist for the gamma subtype of peroxisome proliferator-activated receptors (PPAR-gamma). These compounds exhibit insulin-sensitizing activity (Bergen & Wagner, 2002, Diabetes Tech. & Ther., 4:163-174), and are used in diabetes therapeutics (Savkur et al., Expert Opin Investig Drugs 2006, 15:763-778). Several non-limiting examples of TZD compounds include rosiglitazone, pioglitazone, and troglitazone. It should be appreciated that any TZD compound may be compatible with methods and compositions of the instant invention. In some embodiments of the invention, the TZD compound is rosiglitazone (BRL49563 or Avandia™, Smith-Kline, Brentford, UK).

Aspects of the invention described herein relate, at least in part, to the surprising discovery that treatment of cancer cells (in vitro or in vivo) by contacting the cells with a combination of both an RNase compound such as ranpirnase, and a thiazolidinedione (TZD) compound such as rosiglitazone, enhances the cytotoxic effect of each compound relative to treatment of cancer cells with either compound alone. The combined effect on cancer cells from contact with both an RNase compound and a TZD compounds can be a synergistic cytotoxic effect, i.e., an effect that is greater than the sum of the cytotoxic effects of each compound when administered individually. The combination of an RNase compound and a TZD compound (e.g. a combination of ranpirnase and rosiglitazone) is significantly more active against cancer cells than either drug alone. In addition, the combination has no known or anticipated major toxicities.

In some embodiments of the invention, the combination for contacting cancer cells is the RNase ranpirnase and the TZD compound rosiglitazone. In some embodiments of the invention, one or more additional RNase compounds and/or TZD compounds may be administered to a cell or subject in addition to ranpirnase and rosiglitazone. In some embodiments of the invention, the combination of an RNase and TZD compound is further combined with another cytotoxic agent, such as a chemotherapeutic agent. Non-limiting examples of cytotoxic and/or chemotherapeutic agents include antimetabolites, such as methotrexate, a vinca alkaloid, mitomycin-type antibiotic, bleomycin-type antibiotic, antifolate, colchicine, demecolcine, etoposide, taxane, anthracycline antibiotic, doxorubicin, daunorubicin, caminomycin, epirubicin, idarubicin, mitoxanthrone, 4-demethoxy-daunomycin, 11-deoxydaunorubicin, 13-deoxydaunorubicin, adriamycin-14-benzoate, adriamycin-14-octanoate, adriamycin-14-naphthaleneacetate, amsacrine, carmustine, cyclophosphamide, cytarabine, etoposide, lovastatin, melphalan, topotecan, oxaliplatin, chlorambucil, methotrexate, lomustine, thioguanine, asparaginase, vinblastine, vindesine, tamoxifen, or mechlorethamine, DNA cross-linking agents, such as cisplatin/carboplatin; alkylating agents, such as canbusil; topoisomerase I inhibitors such as dactinomycin; microtubule inhibitors such as taxol (paclitaxol), and the like, as discussed in U.S Patent Publication No. 20070259876.

It should be appreciated that any agent used for cancer treatment, and/or any other cancer treatment method such as radiation or surgery, may be compatible for co-administration, or in combination, with the claimed invention, including any drug that is approved by the Food and Drug Administration (USFDA) or an equivalent organization in another country. Examples of antineoplastic drugs approved by the USFDA appear in US Patent Publication No. 20050261232.

Aspects of the invention relate to contacting a cancer cell with a combination of an RNase compound and a TZD compound each in an amount effective such that the combination decreases the viability of the cancer cell. In some embodiments, contacting the cell occurs in vitro. In some embodiments, a cell that is contacted in vitro may be in a cell culture, and may be a cell that is derived from a cell line, or a cell that has been taken from a subject and cultured. In some embodiments, the cell is a mammalian cell, such as a human cell. In other embodiments it is an animal cell. In some embodiments it is a rodent cell. Some non-limiting examples of human cancer cell lines include: breast cancer cell lines MDA-MB-231, T47D and MCF7; ovarian cancer cell lines SK-OV3 and CA-OV3; prostate cancer cell lines PC3, DU-145 and LNCaP; lung carcinoma cell lines NCI-H292 and N1792 (ATCC, Manassas, Va., USA), and mesothelioma cell lines MP5 and MP6. Further nonlimiting examples of human cancer cell lines available through the National Cancer Institute internet site (dtp.nci.nih) include but are not limited to: CCRF-CEM, HL-60(TB), K-562, MOLT-4, RPMI-8226, SR, A549/ATCC, EKVX, HOP-62, HOP-92, NCI-H226, NCI-H23, NCI-H322M, NCI-H460, NCI-H522, COLO 205, HCC-2998, HCT-116, HCT-15, HT29, KM12, SW-620, SF-268, SF-295, SF-539, SNB-19, SNB-75, U251, LOX IMVI, MALME-3M, M14, SK-MEL-2, SK-MEL-28, SK-MEL-5, UACC-257, UACC-62, IGR-OV1, OVCAR-3, OVCAR-4, OVCAR-5, OVCAR-8, SK-OV-3, 786-0, A498, ACHN, CAKI-1, RXF 393, SN12C, TK-10, UO-31, PC-3, DU-145, MCF7, NCI/ADR-RES, MDA-MB-231/ATCC, HS 578T, MDA-MB-435, BT-549, T-47D, LXFL 529, DMS 114, SHP-77, DLD-1, KM20L2, SNB-78, XF 498, RPMI-7951, M19-MEL, RXF-631, SN12K1, MDA-MB-468, P388, P388/ADR.

In some embodiments, contacting a cancer cell occurs in vivo, in a method of cancer treatment. Methods of the invention may also be used to treat a precancerous condition. As used herein, the term treat, treated, or treating when used with respect to a disorder such as cancer refers to a prophylactic treatment which increases the resistance of a subject to development of the disease or, in other words, decreases the likelihood that the subject will develop the disease as well as a treatment after the subject has developed the disease in order to fight the disease or prevent the disease from becoming worse. The term “treatment” embraces the prevention of cancer and precancerous conditions, and the inhibition and/or amelioration of pre-existing cancers and precancerous conditions. A subject may receive treatment because the subject has been determined to be at risk of developing cancer or a precancerous condition, or alternatively, the subject may have such a disorder. Thus, a treatment may prevent, reduce or eliminate cancer altogether or prevent it from becoming worse.

As used herein, the term “subject” refers to a human or non-human mammal or animal. Non-human mammals include livestock animals, companion animals, laboratory animals, and non-human primates. Non-human subjects also specifically include, without limitation, chickens, horses, cows, pigs, goats, dogs, cats, guinea pigs, hamsters, mink, and rabbits. In some embodiments of the invention, a subject is a patient. As used herein, a “patient” refers to a subject who is under the care of a physician or other health care worker, including someone who has consulted with, received advice from or received a prescription or other recommendation from a physician or other health care worker. A patient is typically a subject having or at risk of having cancer.

As used herein, the term “cancer” refers to an uncontrolled growth of cells that may interfere with the normal functioning of the bodily organs and systems, and includes both primary and metastatic tumors. Primary tumors or cancers that migrate from their original location and seed vital organs can eventually lead to the death of the subject through the functional deterioration of the affected organs. A metastasis is a cancer cell or group of cancer cells, distinct from the primary tumor location, resulting from the dissemination of cancer cells from the primary tumor to other parts of the body. Metastases may eventually result in death of a subject.

As used herein, the term “cancer” includes, but is not limited to, the following types of cancer: breast cancer (including carcinoma in situ), biliary tract cancer; bladder cancer; brain cancer including glioblastomas and medulloblastomas; cervical cancer; choriocarcinoma; colon cancer; endometrial cancer; esophageal cancer; gastric cancer; hematological neoplasms including acute lymphocytic and myelogenous leukemia; T-cell acute lymphoblastic leukemia/lymphoma; hairy cell leukemia; chromic myelogenous leukemia, multiple myeloma; AIDS-associated leukemias and adult T-cell leukemia lymphoma; intraepithelial neoplasms including Bowen's disease and Paget's disease; liver cancer; lung cancer; lymphomas including Hodgkin's disease and lymphocytic lymphomas; mesothelioma, neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Merkel cell carcinoma, Kaposi's sarcoma, basal cell carcinoma, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; and renal cancer including adenocarcinoma and Wilms tumor. Non-limiting examples of precancerous conditions include dysplasia, premalignant lesions, adenomatous colon polyp, and carcinoma in-situ such as Ductal carcinoma in-situ (DCIS), etc. Other cancers that can be treated with methods of the invention will be known to those of ordinary skill in the art. In some embodiments of the invention, the cancer is melanoma. In certain embodiments the cancer is adenocarcinoma. In some embodiments the cancer is a solid tumor cancer. A cancer that may be treated or assayed using methods of the invention also may include breast cancer, lung cancer, prostate cancer, mesothelioma, etc.

Although not wishing to be bound by any particular theory, in some embodiments of the invention, the cancer cell is a cancer cell that expresses the gene Fra-1. Fra-1 belongs to the Fos family of transcription factors, that heterodimerize with proteins from the Jun family of transcription factors, to form the Ap-1 complex, which binds to genes implicated in tumorigenesis (Karin et al., Curr Opin Cell Biol, 1997, p. 240-246; Reddy et al., Am J Physiol (Lung Cell Mol Physiol) 2002, 283, p. L1161-L1178). Fra-1 accumulates in AP-1 complexes during malignant progression of various tumors and has been implicated in maintenance and progression of the transformed state. Signaling pathways implicated in cancer survival such as the ERK1/2, Phosphatidylinositol 3-Kinase (PI3K) and Wnt/beta-Cayenin pathways are also involved in modulating expression of Fra-1 (Ramos-Nino et al., Cancer Res 2002, 62:6065-6069; Kikuchi et al., Biochem Biophys Res Commun, 2000 268(2):243-8; Tulchinsky, Histol Histopathol, 2000, 15(3):921-8; Watts, et al., Oncogene, 1998, 17(26):3493-3498; Vial et al., J Cell Sci 2003, 116(Pt 24):4957-63; Terasawa et al., Genes Cells, 2003 8(3):263-73; Ramos-Nino et al., Am J Respir Cell Mol Biol 2007; Taneyhill et al., BMC Dev Biol, 2004, 4:6). Some embodiments of the invention include cancer cells that express Fra-1, wherein Fra-1 may be regulated through each of these signaling pathways. In certain embodiments of the invention, the cancer cell expresses Fra-1 in a PI3K-dependent manner.

While not wishing to be bound by any particular theory, in some aspects of the invention, the cancer cell is a cancer cell that expresses the gene Survivin. As used herein, the term “high Survivin expression” means any detectable level of survivin in an adult tissue as Survivin is not expressed in normal adult tissues. Survivin is an IAP protein abundantly expressed in fetal tissues (Adida et al., Am J Pathol, 1998, 152:43-49) and neoplasms (Ambrosini et al., Nat Med, 1997, 3:917-921), but undetectable in most normal, terminally differentiated adult tissues (Ambrosini et al., Nat Med, 1997, 3:917-921). High Survivin expression in tumors correlates with more aggressive behavior, decreased response to chemotherapeutic agents, and shortened survival times, as compared with Survivin-negative cancers (reviewed in Johnson et al., Vet Pathol, 2004, 41:599-607). In some embodiments of the invention, the cancer cell expresses Survivin at high levels relative to expression of Survivin in normal adult tissues, (e.g. compared to a normal control adult tissue).

In some embodiments of the invention, the cancer cell that is treated with a combination of an RNase and a TZD compound expresses PPARgamma, while in other embodiments the treated cancer cell does not express PPARgamma.

### Selecting Treatment

In some embodiments, methods of the invention may be used to help select a treatment for a subject with cancer or a precancerous condition. Described herein are methods for assessing effectiveness of a combination of an RNase compound and a TZD compound for treatment of a cancer by contacting a cell of the cancer with a combination of an RNase compound and a TZD compound and determining whether the cell has reduced viability. Reduced viability of a cancer cell following administration of an RNase and TZD compound is interpreted to indicate effectiveness of the combination of the RNase and TZD compound for treatment of the cancer. In some embodiments, the cell is taken from a subject who has cancer. In certain embodiments, sample sources for the cell may include tissues, including but not limited to, lymph tissues; body fluids (e.g., blood, lymph fluid, etc.), cultured cells; cell lines; histological slides; tissue embedded in paraffin; etc. The term “tissue” as used herein refers to both localized and disseminated cell populations including, but not limited to: brain, heart, serum, breast, colon, bladder, epidermis, skin, uterus, prostate, stomach, testis, ovary, pancreas, pituitary gland, adrenal gland, thyroid gland, salivary gland, mammary gland, kidney, liver, intestine, spleen, thymus, bone marrow, trachea, and lung. Invasive and non-invasive techniques can be used to obtain such samples and are well documented in the art. A control cell sample may include a cell, a tissue, or may be a lysate of either. In some embodiments, a control sample may be a sample from a cell or subject that is free of cancer and/or free of a precancerous condition. In some embodiments, a control sample may be a sample that is from a cell or subject that has cancer or a precancerous condition. If the sample is a breast or prostate tissue sample or a breast or prostate cell line, cultured breast of prostate cells, respectively, may be, but need not be, used as a control. It will be understood that controls according to the invention may be, in addition to predetermined values, samples of materials tested in parallel with the experimental materials. Examples include samples from control populations or control samples generated through manufacture to be tested in parallel with the experimental samples.

In some embodiments, factors such as the level of cell growth, proliferation, and/or viability of cells in a sample may be measured. In some embodiments, measurements of cell growth or proliferation can be correlated to levels of cell viability, whereas in other embodiments, cell viability may be measured directly. These factors can be determined in a number of ways when carrying out the various methods of the invention. In one measurement, the level of cell growth, proliferation, or viability of cells in a test sample is measured in relation to a control sample. In some embodiments, a control sample and a test sample may be taken from the same cancer patient. The test sample may be treated with an RNase compound and a TZD compound, while the control sample may not be treated with an RNase compound and a TZD compound. In some embodiments the control sample may be treated with either compound alone or with neither compound. The control and test samples may then be compared for the levels of such characteristics as cell growth, cell proliferation, and/or cell viability of cells in the sample, using art-know methods. For example, if the test sample shows reduced cell growth, and/or proliferation and/or viability, relative to the control sample, then this would be interpreted to mean that cells in the test sample respond to treatment with a combination of an RNase and TZD compound.

In some embodiments, a test sample and a control sample may be from different cancers. In some embodiments, a control sample may be a cancer cell from a type of cancer that is known to respond to treatment with an RNase and TZD compound. In such embodiments, if the test sample responds similarly to the control sample, then the test sample would be interpreted as responding to treatment with an RNase and TZD compound. In other embodiments, a control sample may be a cancer cell from a type of cancer that is known not to respond to treatment with an RNase and TZD compound. In such embodiments, if the test sample responds similarly to the control sample then the test sample would be interpreted as not responding to treatment with an RNase and TZD compound. It will be understood that the interpretation of a comparison between a test sample and a control sample will depend on the nature of both samples.

One possible measurement of the level of cell growth, proliferation, and/or viability of cells in a sample is a measurement of absolute levels of cell growth, proliferation, and/or viability. Another measurement of the level of cell growth, proliferation, or viability of cells in a sample is a measurement of the change in the level of cell growth, proliferation, or viability of cells over time. This may be expressed in an absolute amount or may be expressed in terms of a percentage increase or decrease over time. Methods and assays of the invention may be combined with other methods and assays in determining the level of cell growth, proliferation, and/or viability of cells in a sample, and in determining an optimal treatment strategy for a patient.

In some embodiments, a control value may be a predetermined value, which can take a variety of forms. It can be a single cut-off value, such as a median or mean. It can be established based upon comparative groups, such as in groups having normal amounts of cell growth, proliferation, and/or viability, and groups having abnormal amounts of cell growth, viability, and/or proliferation. For example, in some embodiments a control sample that is taken from a cancer patient and is not treated with an RNase and TZD compound may be considered to have normal levels of cell growth, viability, and proliferation for a cancer cell. In such embodiments, a test sample that is taken from the same cancer patient and treated with a combination of an RNase and TZD compound may be considered to have abnormal levels of cell growth, viability, and/or proliferation.

In another embodiment, non-cancer cells may be considered to have normal levels of cell growth, viability, and proliferation. In this embodiment, a cancer cell taken from a cancer patient, and not treated with a combination of an RNase and TZD compound, may be considered to have abnormal levels of cell growth, viability, and/or proliferation, whereas a cancer cell taken from the same cancer patient and treated with a combination of an RNase and TZD compound may be found to have levels of cell growth, viability, and/or proliferation that approach the normal level, which in such embodiments, would be the levels for a non-cancer cell.

Based at least in part on results of in vitro methods discussed herein, a predetermined value can be arranged. For example, test samples and the subjects from which the samples were extracted, are divided equally (or unequally) into groups, such as a low-response group, a medium-response group and a high-response group, where response refers to response of the sample from each group to treatment with a combination of an RNase and TZD compounds using methods described herein. Test samples and subjects may be divided into quadrants or quintiles, the lowest quadrant or quintile being individuals with the lowest response and the highest quadrant or quintile being individuals with the highest response. Individuals with the highest level of response to the treatment would be considered the most likely to respond to the treatment. However individuals in low and medium response groups may also be found to respond to the treatment.

The predetermined value, of course, will depend upon the particular population selected. For example, an apparently healthy population will have a different ‘normal’ range than will a population that is known to have a cancer. In addition, values may be different for different cancers, or for different populations or individuals. Accordingly, the predetermined value selected may take into account the category in which an individual or cell falls. Appropriate ranges and categories can be selected with no more than routine experimentation by those of ordinary skill in the art. As used herein, “abnormal” means not normal as compared to a control. By abnormally high or low it is meant high or low relative to a selected control.

As mentioned elsewhere herein, it is also possible to use measurements of cell growth, proliferation, and/or viability to monitor changes in the levels of these factors over time in a cell sample. For example, in some embodiments it is expected that treatment of a cancer cell with a combination of an RNase and a TZD compound will lead to a decrease in levels of cell growth, proliferation, and/or viability relative to a control sample of a cancer cell that is not treated with a combination of an RNase and a TZD compound, or a control sample of a cancer cell that is not responsive to a combination of an RNase and a TZD compound. Accordingly, one can monitor levels of cell growth, proliferation, and/or viability over time to determine if there is a change in the levels of these factors in a subject or in a cell culture. In some embodiments, changes in levels of cell growth, proliferation, and/or viability greater than 0.1% may be considered to indicate effectiveness of the treatment on the levels of these factors. In some embodiments, the reduction in levels of cell growth, viability and/or proliferation, which indicate effectiveness of the treatment on these factors, is a reduction greater than 0.2%, greater than 0.5%, greater than 1.0%, 2.0%, 3.0%, 4.0%, 5.0%, 7.0%, 10%, 15%, 20%, 25%, 30%, 40%, 50%, 60%, 70%, 80%, 90%, 95%, 99%, or more, including each percentage in between these values. Decreases in the levels of cell growth, proliferation, and/or viability of cancer cells over time may indicate a change in responsiveness to treatment in a sample or subject. To make a determination of a change in responsiveness to treatment in a subject over time, multiple samples may be obtained from the subject at different times and the samples tested for levels of cell growth, proliferation, and/or viability of cancer cells. Resulting values may be compared to each other as a measure of change over time.

Methods of selecting a treatment may be useful to assess and/or adjust treatment of subjects already receiving a drug or therapy (e.g., radiation treatment or surgery) for treating cancer or a precancerous condition. Based on the determination of the response of a cancer cell to administration of an RNase and TZD compound, it may be appropriate to alter a therapeutic regimen for a subject. For example, determination that a cancer cell responds to administration of an RNase and TZD compound in a subject who has received or is receiving a cancer or precancerous-condition treatment may indicate that the treatment regimen should be adjusted (e.g., the dose or frequency of dosing, increased, new treatment initiated, etc.). For example, a reduction in cancer cell viability after contact with a combination of an RNase and TZD compound indicates that the cancer is responsive to the treatment and that the treatment may be useful to treat that cancer. Different parameters can be assessed to determine appropriate optimized treatment regimens for a given patient's cancer.

### Administration

Aspects of the invention relate to co-administering therapeutic compounds. Compounds of the invention may be co-administered in effective amounts. As used herein, co-administration refers to administration of at least two compounds in such a way relative to each other to result in a desired effect. Co-administration can include embodiments where compounds of the invention are administered physically and/or temporally together or separately. When co-administered, the effective amount of a compound to achieve a desired result may be different from the effective amount of the compound administered alone. An effective amount of a compound of the invention, when co-administered with a second compound of the invention, is a dosage of the compound sufficient to provide a medically desirable result. Typically, an effective amount of each compound to be administered in combination to achieve a desired result will be determined in clinical trials, establishing effective doses of the compounds for a test population versus a control population in a blind study. An effective amount of a combination of compounds (which may also be referred to herein as a pharmaceutical or therapeutic compound) means that amount of the compound necessary to delay the onset of, inhibit the progression of or halt altogether the onset or progression of the particular condition (e.g., a cancer) being treated—when administered in combination with another therapeutic compound of the combination. An effective amount may be an amount that, when in combination with the other compound of the combination, reduces one or more signs or symptoms of the condition (e.g., a cancer). Other assays will be known to one of ordinary skill in the art and can be employed for measuring the level of the response to a treatment. The amount of a treatment may be varied for example by increasing or decreasing the amount of a therapeutic compound or multiple compounds, by changing the combination of therapeutic compounds administered, by changing the route of administration, by changing the dosage timing and so on. When administered to a subject, effective amounts will depend, of course, on the particular condition being treated (e.g., a cancer), the severity of the condition, individual subject parameters including age, physical condition, size and weight, concurrent treatment, frequency of treatment, and the mode of administration. These factors are well known to those of ordinary skill in the art and can be addressed with no more than routine experimentation.

A pharmaceutical compound dosage may be adjusted by the individual physician or veterinarian, particularly in the event of any complication. A therapeutically effective amount typically varies from 0.01 mg/kg to about 1000 mg/kg, preferably from about 0.1 mg/kg to about 200 mg/kg, and most preferably from about 0.2 mg/kg to about 20 mg/kg, in 1 or more dose administrations daily, weekly, every two weeks, every three weeks, monthly, etc.

The absolute amount of each compound of combination administered will depend upon a variety of factors, including the material selected for administration, whether the administration is in single or multiple doses, and individual subject parameters including age, physical condition, size, weight, and the stage of the disease or condition. These factors are well known to those of ordinary skill in the art and can be addressed with no more than routine experimentation.

Pharmaceutical compounds of the invention may be administered alone, in combination with each other, and/or in combination with other drug therapies, or other treatment regimens that are administered to subjects with cancer.

Actual dosage levels of active ingredients of the invention, for example an RNase compound or a TZD compound, can be varied to obtain an amount that is effective to achieve the desired therapeutic response for a particular subject, compound, and mode of administration. The selected dosage level depends upon the activity of the particular compound, the route of administration, the severity of the condition being treated, the condition, and prior medical history of the subject being treated. However, it is within the skill of the art to start doses of the compound at levels lower than required to achieve the desired therapeutic effort and to gradually increase the dosage until the desired effect is achieved. In some embodiments, lower dosages would be required for co-administration of multiple compounds than for administration of single compounds (e.g. co-administration of an RNase and a TZD compound, or co-administration of a composition containing both an RNase compound and a TZD compound, may require lower dosages than administration of either compound singly or a composition containing either compound singly). In other embodiments of the invention, dosages for administration of the compounds in a combination may be the same as or higher than the amount required for administration of one of the compounds alone.

Compounds of the invention may be delivered to a subject on an as needed basis. In some embodiments of the invention, a physician or other health care worker may select a delivery schedule. In other embodiments of the invention, the compounds are administered on a routine schedule. A “routine schedule” as used herein, refers to a predetermined designated period of time. The routine schedule may encompass periods of time which are identical or which differ in length, as long as the schedule is predetermined. For instance, the routine schedule may involve administration of the composition on a daily basis, every two days, every three days, every four days, every five days, every six days, a weekly basis, two-week basis, a three-week basis, a monthly basis or any set number of days or weeks there-between, every two weeks, three weeks, four weeks, two months, three months, four months, five months, six months, seven months, eight months, nine months, ten months, eleven months, twelve months, etc. Alternatively, the predetermined routine schedule may involve administration of the composition on a daily basis for the first week, followed by a weekly or monthly basis for several months, and then every three months after that. These regimens are exemplary and those of ordinary skill in the art will recognize that any number of administration regimens may be used to result in optimal treatment of a cancer. Any particular combination would be covered by the routine schedule as long as it is determined ahead of time that the appropriate schedule involves administration on a certain day. It will be understood that the schedule may be adjusted due to the needs of the patient.

In some embodiments of the invention, the RNase compound and the TZD compound are co-administered in a single pharmaceutical composition. In other embodiments, the RNase compound and the TZD compound are administered separately. It should be appreciated that when the RNase compound and TZD compound are administered separately, the compounds may or may not be administered according to the same routine schedule. In some embodiments, the schedule for administering the two compounds coincides and/or overlaps. In other embodiments, the schedule for administering the two compounds does not coincide or overlap, meaning that the compounds are administered following independent schedules. For example, an RNase compound may be administered weekly and the TZD compound may be administered every two weeks. The compounds are still said to be administered in combination (co-administered) even if each has a different administration schedules than the other, as long as the compounds are administered in a manner that results in a more-than-additive effect on cell growth, proliferation, and/or viability of cancer cells, than either of the compounds when administered not in combination with the other compound. A non-limiting example of a regimen of co-administration may include intravenous administration of ranpirnase every two weeks for three cycles and concomitant administration of rosiglitazone by mouth daily for six months. An alternative co-administration example can include administration of one rosiglitazone dose by mouth coupled with intravenous administration of ranpirnase every day for a week. It will be understood by those of ordinary skill in the art that any method of co-administration is acceptable as long as the method allows a synergistic effect of the two compounds to occur in the subject or to occur in conjunction with the cells to be treated. An optimal schedule for co-administering the two compounds may be determined empirically as would be understood by one of skill in the art.

The compounds of the invention, or compositions comprising the compounds of the invention, can be administered to a subject by any suitable route. For example, the compounds or compositions can be administered orally, including sublingually, rectally, parenterally, intracisternally, intravaginally, intraperitoneally, topically and transdermally (as by powders, ointments, or drops), bucally, or nasally. The term “parenteral” administration as used herein refers to modes of administration other than through the gastrointestinal tract, which include intravenous, intramuscular, intraperitoneal, intrasternal, intramammary, intraocular, retrobulbar, intrapulmonary, intrathecal, subcutaneous and intraarticular injection and infusion. Surgical implantation also is contemplated, including, for example, embedding a composition of the invention in the body such as, for example, in the brain, in the abdominal cavity, under the splenic capsule, or in the cornea.

It will be understood that the RNase compound and the TZD compound may or may not be administered by the same route. For example in some embodiments, both compounds may be administered orally. In certain embodiments one compound may be administered orally, and the other compound may be administered intravenously. In certain embodiments, both compounds may be administered intravenously.

Dosage forms for topical administration of a composition of this invention include powders, sprays, ointments, and inhalants as described herein. The composition is mixed under sterile conditions with a pharmaceutically acceptable carrier and any needed preservatives, buffers, or propellants that may be required.

Pharmaceutical compositions of the invention for parenteral injection comprise pharmaceutically acceptable sterile aqueous or nonaqueous solutions, dispersions, suspensions, or emulsions, as well as sterile powders for reconstitution into sterile injectable solutions or dispersions just prior to use. Examples of suitable aqueous and nonaqueous carriers, diluents, solvents, or vehicles include water, ethanol, polyols (such as glycerol, propylene glycol, polyethylene glycol, and the like), and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

These compositions also can contain adjuvants such as preservatives, wetting agents, emulsifying agents, and dispersing agents. Prevention of the action of microorganisms can be ensured by the inclusion of various antibacterial and antifungal agents, for example, paraben, chlorobutanol, phenol sorbic acid, and the like. It also may be desirable to include isotonic agents such as sugars, sodium chloride, and the like. Prolonged absorption of the injectable pharmaceutical form can be brought about by the inclusion of agents that delay absorption, such as aluminum monostearate or gelatin.

In some cases, in order to prolong the effect of the composition, it is desirable to slow the absorption of the composition from subcutaneous or intramuscular injection. This result can be accomplished by the use of a liquid suspension of crystalline or amorphous materials with poor water solubility. The rate of absorption of the composition then depends upon its rate of dissolution, which, in turn, may depend upon crystal size and crystalline form. Alternatively, delayed absorption of a parenterally administered composition from is accomplished by dissolving or suspending the composition in an oil vehicle.

Injectable depot forms are made by forming microencapsule matrices of the composition in biodegradable polymers such a polylactide-polyglycolide. Depending upon the ratio of composition to polymer, and the nature of the particular polymer employed, the rate of composition release can be controlled. Examples of other biodegradable polymers include poly(orthoesters) and poly(anhydrides). Depot injectable formulations also are prepared by entrapping the drug in liposomes or microemulsions that are compatible with body tissue.

The injectable formulations can be sterilized, for example, by filtration through a bacterial- or viral-retaining filter, or by incorporating sterilizing agents in the form of sterile solid compositions, which can be dissolved or dispersed in sterile water or other sterile injectable medium just prior to use.

The invention provides methods for oral administration of a pharmaceutical composition of the invention. Oral solid dosage forms are described generally in Remington's Pharmaceutical Sciences, 18th Ed., 1990 (Mack Publishing Co. Easton Pa. 18042) at Chapter 89. Solid dosage forms for oral administration include capsules, tablets, pills, powders, troches or lozenges, cachets, pellets, and granules. Also, liposomal or proteinoid encapsulation can be used to formulate the present compositions (as, for example, proteinoid microspheres reported in U.S. Pat. No. 4,925,673). As is known in the art, liposomes generally are derived from phospholipids or other lipid substances. Liposomes are formed by mono- or multi-lamellar hydrated liquid crystals that are dispersed in an aqueous medium. Any nontoxic, physiologically acceptable, and metabolizable lipid capable of forming liposomes can be used. The present compositions in liposome form can contain, in addition to a compound of the present invention, stabilizers, preservatives, excipients, and the like. The preferred lipids are the phospholipids and the phosphatidyl cholines (lecithins), both natural and synthetic. Methods to form liposomes are known in the art. See, for example, Prescott, Ed., Methods in Cell Biology, Volume XIV, Academic Press, New York, N.Y. (1976), p 33, et seq. Liposomal encapsulation may include liposomes that are derivatized with various polymers (e.g., U.S. Pat. No. 5,013,556). In general, the formulation includes a composition of the invention and inert ingredients which protect against degradation in the stomach and which permit release of the biologically active material in the intestine.

In such solid dosage forms, the composition is mixed with, or chemically modified to include, a least one inert, pharmaceutically acceptable excipient or carrier. The excipient or carrier preferably permits (a) inhibition of proteolysis, and (b) uptake into the blood stream from the stomach or intestine. In one embodiment, the excipient or carrier increases uptake of the composition of the invention, overall stability of the composition, and/or circulation time of the composition in the body. Excipients and carriers include, for example, sodium citrate, or dicalcium phosphate, and/or (a) fillers or extenders such as starches, lactose, sucrose, glucose, cellulose, modified dextrans, mannitol, and silicic acid, as well as inorganic salts such as calcium triphosphate, magnesium carbonate and sodium chloride, and commercially available diluents such as FAST-FLO®, EMDEX®, STA-RX 1500®, EMCOMPRESS® and AVICEL®, (b) binders such as, for example, methylcellulose ethylcellulose, hydroxypropylmethyl cellulose, carboxymethylcellulose, gums (e.g., alginates, acacia), gelatin, polyvinylpyrrolidone, and sucrose, (c) humectants, such as glycerol, (d) disintegrating agents, such as agar-agar, calcium carbonate, potato or tapioca starch, alginic acid, certain silicates, sodium carbonate, starch including the commercial disintegrant based on starch, EXPLOTAB®, sodium starch glycolate, AMBERLITE®, sodium carboxymethylcellulose, ultramylopectin, gelatin, orange peel, carboxymethyl cellulose, natural sponge, bentonite, insoluble cationic exchange resins, and powdered gums such as agar, karaya or tragacanth; (e) solution retarding agents such a paraffin, (f) absorption accelerators, such as quaternary ammonium compounds and fatty acids including oleic acid, linoleic acid, and linolenic acid (g) wetting agents, such as, for example, cetyl alcohol and glycerol monosterate, anionic detergent surfactants including sodium lauryl sulfate, dioctyl sodium sulfosuccinate, and dioctyl sodium sulfonate, cationic detergents, such as benzalkonium chloride or benzethonium chloride, nonionic detergents including lauromacrogol 400, polyoxyl 40 stearate, polyoxyethylene hydrogenated castor oil 10, 50 and 60, glycerol monostearate, polysorbate 40, 60, 65, and 80, sucrose fatty acid ester, methyl cellulose and carboxymethyl cellulose; (h) absorbents, such as kaolin and bentonite clay, (i) lubricants, such as talc, calcium sterate, magnesium stearate, solid polyethylene glycols, sodium lauryl sulfate, polytetrafluoroethylene (PTFE), liquid paraffin, vegetable oils, waxes, CARBOWAX® 4000, CARBOWAX® 6000, magnesium lauryl sulfate, and mixtures thereof; (j) glidants that improve the flow properties of the drug during formulation and aid rearrangement during compression that include starch, talc, pyrogenic silica, and hydrated silicoaluminate. In the case of capsules, tablets, and pills, the dosage form also can comprise buffering agents.

Solid compositions of a similar type also can be employed as fillers in soft and hard-filled gelatin capsules, using such excipients as lactose or milk sugar, as well as high molecular weight polyethylene glycols and the like.

The solid dosage forms of tablets, dragees, capsules, pills, and granules can be prepared with coatings and shells, such as enteric coatings and other coatings well known in the pharmaceutical formulating art. They optionally can contain opacifying agents and also can be of a composition that they release the active ingredients(s) only, or preferentially, in a part of the intestinal tract, optionally, in a delayed manner. Exemplary materials include polymers having pH sensitive solubility, such as the materials available as EUDRAGIT® Examples of embedding compositions that can be used include polymeric substances and waxes.

The composition of the invention also can be in microencapsulated form, if appropriate, with one or more of the above-mentioned excipients.

Liquid dosage forms for oral administration include pharmaceutically acceptable emulsions, solutions, suspensions, syrups, and elixirs. In addition to the composition of the invention, the liquid dosage forms can contain inert diluents commonly used in the art, such as, for example, water or other solvents, solubilizing agents and emulsifiers, such as ethyl alcohol, isopropyl alcohol ethyl carbonate ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydroflirfuryl alcohol, polyethylene glycols, fatty acid esters of sorbitan, and mixtures thereof.

Besides inert diluents, the oral compositions also can include adjuvants, such as wetting agents, emulsifying and suspending agents, sweetening, coloring, flavoring, and perfuming agents. Oral compositions can be formulated and further contain an edible product, such as a beverage.

Suspensions, in addition to the composition of the invention, can contain suspending agents such as, for example ethoxylated isostearyl alcohols, polyoxyethylene sorbitol and sorbitan esters, microcrystalline cellulose, aluminum metahydroxide, bentonite, agar-agar, tragacanth, and mixtures thereof.

Also contemplated herein is pulmonary delivery of the composition of the invention. The composition is delivered to the lungs of a mammal while inhaling, thereby promoting the traversal of the lung epithelial lining to the blood stream. See, Adjei et al., Pharmaceutical Research 7:565-569 (1990); Adjei et al., International Journal of Pharmaceutics 63:135-144 (1990) (leuprolide acetate); Braquet et al., Journal of Cardiovascular Pharmacology 13 (suppl. 5): s. 143-146 (1989) (endothelin-1); Hubbard et al., Annals of Internal Medicine 3:206-212 (1989) (α1-antitrypsin); Smith et al., J. Clin. Invest. 84:1145-1146 (1989) (α1-proteinase); Oswein et al., “Aerosolization of Proteins,” Proceedings of Symposium on Respiratory Drug Delivery II, Keystone, Colo., March, 1990 (recombinant human growth hormone); Debs et al., The Journal of Immunology 140:3482-3488 (1988) (interferon-7 and tumor necrosis factor α) and Platz et al., U.S. Pat. No. 5,284,656 (granulocyte colony stimulating factor).

Contemplated for use in the practice of this invention are a wide range of mechanical devices designed for pulmonary delivery of therapeutic products, including, but not limited to, nebulizers, metered dose inhalers, and powder inhalers, all of which are familiar to those skilled in the art.

Some specific examples of commercially available devices suitable for the practice of the invention are the ULTRAVENT® nebulizer, manufactured by Mallinckrodt, Inc., St. Louis, Mo.; the ACORN II® nebulizer, manufactured by Marquest Medical Products, Englewood, Colo.; the VENTOL® metered dose inhaler, manufactured by Glaxo Inc., Research Triangle Park, N.C.; and the SPINHALER® powder inhaler, manufactured by Fisons Corp., Bedford, Mass.

All such devices require the use of formulations suitable for the dispensing of a composition of the invention. Typically, each formulation is specific to the type of device employed and can involve the use of an appropriate propellant material, in addition to diluents, adjuvants, and/or carriers useful in therapy.

The composition may be prepared in particulate form, preferably with an average particle size of less than 10 μm, and most preferably 0.5 to 5 μm, for most effective delivery to the distal lung.

Carriers include carbohydrates such as trehalose, mannitol, xylitol, sucrose, lactose, and sorbitol. Other ingredients for use in formulations may include lipids, such as DPPC, DOPE, DSPC and DOPC, natural or synthetic surfactants, polyethylene glycol (even apart from its use in derivatizing the inhibitor itself), dextrans, such as cyclodextran, bile salts, and other related enhancers, cellulose and cellulose derivatives, and amino acids.

In addition, the use of liposomes, microcapsules or microspheres, inclusion complexes, or other types of carriers is contemplated.

Formulations suitable for use with a nebulizer, either jet or ultrasonic, typically comprise a composition of the invention dissolved in water at a concentration of about 0.1 to 25 mg of biologically active protein per mL of solution. The formulation also can include a buffer and a simple sugar (e.g., for protein stabilization and regulation of osmotic pressure). The nebulizer formulation also can contain a surfactant to reduce or prevent surface-induced aggregation of the inhibitor composition caused by atomization of the solution in forming the aerosol.

Formulations for use with a metered-dose inhaler device generally comprise a finely divided powder containing the composition of the invention suspended in a propellant with the aid of a surfactant. The propellant can be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid also can be useful as a surfactant.

Formulations for dispensing from a powder inhaler device comprise a finely divided dry powder containing the composition of the invention and also can include a bulking agent, such as lactose, sorbitol, sucrose, mannitol, trehalose, or xylitol, in amounts that facilitate dispersal of the powder from the device, e.g., 50 to 90% by weight of the formulation.

Nasal delivery of the composition of the invention also is contemplated. Nasal delivery allows the passage of the composition to the blood stream directly after administering the therapeutic product to the nose, without the necessity for deposition of the product in the lung. Formulations for nasal delivery include those with dextran or cyclodextran. Delivery via transport across other mucous membranes also is contemplated.

Compositions for rectal or vaginal administration are preferably suppositories that can be prepared by mixing the composition of the invention with suitable nonirritating excipients or carriers, such as cocoa butter, polyethylene glycol, or suppository wax, which are solid at room temperature, but liquid at body temperature, and therefore melt in the rectum or vaginal cavity and release the active compounds.

### Kits

Also within the scope of the invention are kits comprising the compounds of the invention and instructions for use. Kits of the invention may be useful for determining a treatment regimen for cancer or a precancerous condition. An example of such a kit may include an RNase compound, a TZD compound, and instructions for use of the two compounds for determining whether the combination of the two compounds can be used as a treatment regimen for the cancer. Kits of the invention may also be useful for treating cancer. An example of such a kit may include an RNase compound, a TZD compound, and instructions for use of a combination of the compounds for treating the cancer. A kit of the invention can include a description of use of the composition for participation in any biological or chemical mechanism disclosed herein. Kits can further include a description of activity of the condition in treating the pathology, as opposed to the symptoms of the condition. That is, a kit can include a description of use of the compositions as discussed herein. A kit also can include instructions for use of a combination of two or more compositions of the invention, or instruction for use of a combination of a composition of the invention and one or more other compounds indicated for determining a treatment regimen for cancer or for treatment of a cancer. Instructions also may be provided for administering the composition by any suitable technique as previously described.

The kits described herein may also contain one or more containers, which may contain a composition and other ingredients as previously described. The kits also may contain instructions for mixing, diluting, and/or administering or applying the compositions of the invention in some cases. The kits also can include other containers with one or more solvents, surfactants, preservative and/or diluents (e.g., normal saline (0.9% NaCl), or 5% dextrose) as well as containers for mixing, diluting or administering the components in a sample or to a subject in need of such treatment.

The compositions of the kit may be provided as any suitable form, for example, as liquid solutions or as dried powders. When the composition provided is a dry powder, the composition may be reconstituted by the addition of a suitable solvent, which may also be provided. In embodiments where liquid forms of the composition are used, the liquid form may be concentrated or ready to use. The solvent will depend on the composition and the mode of use or administration. Suitable solvents for drug compositions are well known, for example as previously described, and are available in the literature. The solvent will depend on the composition and the mode of use or administration.

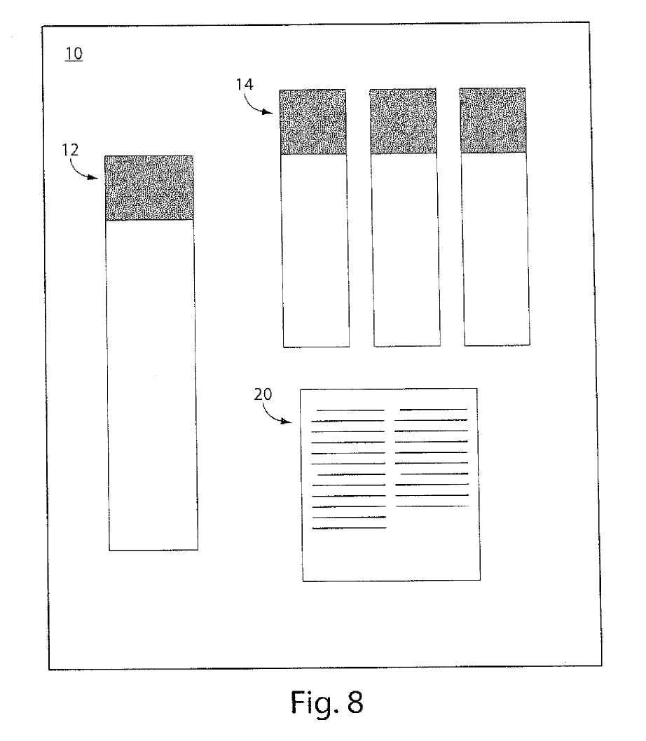

An example of a kit useful according to the invention is shown in FIG. 8. The kit (10) shown in FIG. 8 includes a set of containers for housing compounds such as an RNase or a TZD compound (12) and other compounds (14) as well as instructions (20).

The present invention is further illustrated by the following Examples, which in no way should be construed as further limiting. The entire contents of all of the references (including literature references, issued patents, published patent applications, and co-pending patent applications) cited throughout this application are hereby expressly incorporated herein by reference.

### EXAMPLES

### Example 1

### A Novel Combination: Ranpirnase and Rosiglitazone Induce a Synergistic Anti-Apoptotic Effect by Down-Regulating Fra-1 and Survivin in Cancer Cells

The effect of a novel drug combination consisting of ranpirnase and rosiglitazone was investigated for its effects on the viability of cancer cells. This drug combination was found to synergistically decreases cell viability in seven out of twelve cancer cell lines. Flow cytometry techniques determined that the drug combination causes increases in hypodiploid (subG₀/G₁) cells and apoptosis (Annexin V-positive cells). Growth curves further demonstrated a reduction in growth after treatment with the combination in some cancer cell lines. Knock-down of Fra-1 increases cell killing by ranpirnase in a dose-dependent manner, but not by rosiglitazone. The drug combination does not have a synergistic effect on killing in Fra-1 knock-down cells, demonstrating that Fra-1 modulation accounts in part for the synergism. Antineoplastic drug efficacy depends, in part, on the survival signaling pathways activated in specific tumor cells. The novel drug combination of ranpirnase and rosiglitazone is a combination for treatment of cancers including cancers with increased PI3K-dependent Fra-1 expression or Survivin.

### BACKGROUND

Fra-1 and Survivin are key signaling proteins in the development and progression of various cancers. Fos family member Fra-1 accumulates in transcription factor activator protein-1 (AP-1) complexes during the malignant progression of several tumors, and has been implicated in both the maintenance and progression of the transformed state. Fra-1's causal role in cellular transformation has been documented in several systems, including esophagus [1], and breast [2,3]. Ectopic expression of Fra-1 in vitro increases the cell motility and metastatic behavior of mammary adenocarcinoma cells [4]. Also, a higher level of Fra-1 expression is essential for the v-mos induced transformation of thyroid cells [5]. A broad overexpression of Fra-1, but not other AP-1 components, induces some lung tumors in mice [6]. Fra-1 mRNA is also highly expressed in NNK-induced lung tumors, as compared to control lung tissue [7]. Lastly, Fra-1 is a predominant component of the AP-1 complex in asbestos-induced mesothelioma and proliferating rat mesothelioma cells, while overexpression of a dominant negative Fra-1 mutant inhibits the growth of these cells in soft agar [8]. Survivin is an IAP protein abundantly expressed in fetal tissues [9] and neoplasms [10], but undetectable in most normal, terminally differentiated adult tissues [10]. High Survivin expression by tumors correlates with more aggressive behavior, decreased response to chemotherapeutic agents, and shortened survival times, as compared with Survivin-negative cancers (reviewed in [11]).

Here, a novel drug combination (ranpirnase and rosiglitazone) is presented that is capable of down-regulating both Fra-1 and Survivin in several cancer cell lines. The anticancer effect of ranpirnase (reviewed in [12]) has been documented both in vitro [13-19] and in vivo [14, 16, 18, 20]. This amphibian ribonuclease drug has low toxicity to normal cells and shows effectiveness against cancer cells. Previously, it was reported that in malignant mesothelioma cells with increased kinase activity levels of AKT, both LY294002 and wortmannin (two inhibitors of the phosphatidylinositol 3′-Kinase (PI3K)/AKT pathway) act cooperatively with ranpirnase to inhibit cell growth [21]. The combination of the TZD rosiglitazone and ranpirnase has now been investigated. Specifically, the combination of ranpirnase and rosiglitazone has now been tested in several cancer cell lines, showing that two PI3K downstream targets, Fra-1 [24] and Survivin [25], are down-regulated by the combination. Furthermore, a synergistic, apoptotic effect of ranpirnase and rosiglitazone has been demonstrated in cancer cell lines directly associated with the expression of Fra-1. This drug combination provides an important chemotherapeutic alternative for treatment of cancers.

### Materials and Methods

### Cell Lines

The breast cancer cell lines MDA-MB-231, T47D and MCF7; the ovarian cancer cell lines SK-OV3 and CA-OV3; the prostate cancer cell lines PC3, DU-145 and LNCaP; the lung carcinoma cell lines NCI-H292 and N1792 (all from ATCC, Manassas, Va., USA); and the mesothelioma cell lines MP5 and MP6 (Dr. Harvey Pass, New York University) were maintained in frozen stocks. Cells were incubated at 37° C. in 5% CO₂ until approximately 80-90% confluency in DMEM medium (GIBCO BRL, NY) containing 5% fetal bovine serum (FBS) and 1 g/L of glucose. Cells were then starved O/N in DMEM containing 0.5% FBS before treatments with ranpirnase, rosiglitazone alone or in combination for 2 or 6 days, and then collected. When indicated, media were changed again after treatment to DMEM/F12 containing 0.5% FBS and insulin 1 μM to activate the PI3K pathway.

### Small Molecule Inhibitors and Chemicals

Stock solution of the PI3K's small molecular inhibitor LY294002 was diluted in dimethyl sulfoxide (DMSO) and used at effective nontoxic concentrations (20 μM) [24] (Calbiochem, La Jolla, Calif.). Ranpirnase (Onconase™, kindly provided by Dr. Kuslima Shogen, AlfaCell Corporation, Bloomfield, N.J.), was used at three concentrations (0.1, 1, and 10 μg/ml medium), and was prepared as aliquots in medium from a lyophilized stock solution subsequently frozen at −20° C. Rosiglitazone (Cayman Chemical, Ann Arbor, Mich.) was dissolved in DMSO and used at concentrations of 10 and 20 μM (<LD50 for all cells tested). GW9662 (Cayman Chemical), an irreversible PPARγ antagonist, was prepared in the same manner, and used at a concentration of 2 μM. All untreated control cells received DMSO in medium.

### Methoxy-Tetrazolium Salt (MTS) Assay for Cell Viability

Assays were performed on 96-well microtiter plates after plating of 7.7×10⁴ cells/well. Cells were then cultured for 24 h in complete medium before changing to medium containing 0.5% FBS with ranpirnase at 0.1, 1 or 10 μg/ml, rosiglitazone at 10 or 20 μM, their six combinations, or DMSO-containing medium (solvent control). Cell viability was measured by the colorimetric methoxy-tetrazolium salt (MTS) Assay, CellTiter 96 Aqueous One Solution Cell Proliferation Assay (Promega), per the manufacturer's recommendations. MTS is a tetrazolium salt that undergoes a color change caused by its bioreduction of MTS into a water-soluble formazan. The conversion of MTS into the aqueous-soluble formazan is accomplished by dehydrogenase enzymes found in active mitochondria, with reaction occurring only in living cells. The quantity of formazan product measured by the amount of 490-nm light absorbance is directly proportional to the number of living cells in culture. Briefly, 20 μl of MTS reagent was added per well, and plates incubated at 37° C. for 2-3 h. Finally, the absorbance of each well was read at 490 nm and 650 nm. ΔOD from these two wavelengths were reported as the corrected viability. Fold changes were calculated with respect to the control as a measure of cell viability.

### Flow Cytometry

Near confluent cells were maintained in complete medium containing 0.5% FCS overnight before addition of ranpirnase at 1 μg/ml, rosiglitazone 20 μM, their combination, or DMSO-containing medium (solvent control). At 48 h, medium was removed and adherent cells harvested by trypsinization. Combined cells were resuspended at 10⁶/ml in staining solution (50 μg/ml propidium iodide, 0.1% Triton X-100, and 32 μg/ml RNAse A) in phosphate-buffered saline and incubated for 30 min at 37° C. before analysis of 10,000 cells/group/time point in triplicate. The distribution of cells, including cells with a hypodiploid DNA content indicative of apoptosis or necrosis, was determined using a Coulter Epics Elite flow cytometer and appropriate software, as described previously [29]. To determine number of apoptotic cells, cells were stained with Annexin V and propidium iodide (PI) in the dark for 15 min and 5,000 events per sample, analyzed by flow cytometry as described above. For staining, cell pellets were suspended in 93 μl of 1× binding buffer [10 mM Hepes/NaOH (pH 7.4), 140 mM NaCl, 2.5 mM CaCl₂], 5 μl of PI at a final concentration of 2.5 μg/ml (Sigma), and 2 μl of FITC labeled-Annexin V (BD Bioscience, San Jose, Calif.). Cells with annexin V-positive staining were scored as apoptotic.

### Growth Curves

Cells (N=2-3 plates/group/time point) were plated at approximately 1×10³ cells per 6-well plate in complete medium, allowed to attach for 24 h, and then treated with inhibitors at different time points. Cells were removed by trypsinization, and aliquots counted using a hemocytometer to determine total cell number.

### Western Blot Analyses

Near confluent MM cells were washed 3× with cold phosphate-buffered saline (PBS) before centrifugation at 14,000 rpm for 1 min. The pellet was resuspended in lysis buffer [20 mM Tris (pH 7.4), 1% Triton X-100, 10% glycerol, 137 mM NaCl, 2 mM EDTA, 25 mM β-glycerophosphate, 1 mM Na₃VO₄, 2 mM pyrophosphate, 1 mM PMSF, 10 μg/ml leupeptin, 1 mM DTT, 10 mM NaF, 1% aprotinin], incubated at 4° C. for 15 min, and centrifuged at 14,000 rpm for 20 min. The amount of protein in each supernatant was determined using the Bio-Rad protein assay (Bio-Rad, Hercules, Calif.). Thirty μg protein in sample buffer [62.5 mM Tris-HCl (pH 6.8), 2% sodium dodecyl sulfate (SDS), 10% glycerol, 50 mM dithiothreitol, 0.1% w/v bromophenol blue] was electrophoresed in 10% SDS-polyacrylamide gel, and transferred to nitrocellulose using a semi-dry transfer apparatus (Ellard Instrumentation, Ltd., Seattle, Wash.). Blots were blocked in buffer [Tris-buffered saline (TBS) containing 5% nonfat dry milk plus 0.1% Tween-20 (Sigma)] for 1 h, washed 3× for 5 min each in TBS/0.1% Tween-20, and incubated at 4° C. with an antibody specific to Fra-1 (R-20) at a 1:500 dilution or Survivin (D8) at 1:100 dilution (Santa Cruz Biotechnology, Santa Cruz, Calif.). Blots were then washed 3× with TBS/0.1% Tween-20 and incubated with a specific peroxidase-conjugated secondary antibody for 1 h. After washing blots 3× in TBS/0.1% Tween-20, protein bands were visualized with the LumiGlo enhanced chemiluminescence detection system (Kirkgaard and Perry Laboratories, Gaithersburg, Md.) and quantitated by densitometry [30]. Blots were reprobed with an antibody to α-Tubulin (Santa Cruz Biotechnology, Santa Cruz, Calif.) to validate equal loading between lanes.

### Constructs and Transfection Techniques

siFra-1 RNA interference (RNAi) duplexes were constructed from sequence information on mature mRNA extracted from the EST database available through the National Center for Biotechnology Information internet site using the open frame region from the cDNA sequence of exon 2 of the Fra-1 gene. The siRNA pool sequences targeting Fra-1 corresponded to the 107-126, 124-143, 230-249 coding regions relative to the first nucleotide of the start codon. The sequences were BLAST-searched (NCBI database) against EST libraries to ensure the specificity of the siRNA molecule. The siRNA duplexes or a scramble control were transfected into cancer lines using Lipofectamine 2000 (Invitrogen) as recommended by the manufacturer. Cells were incubated with complexes overnight, and the medium was replaced the next day. Cells were allowed to recover for 48 hours before treatments.

### SYBR Green Real Time Quantitative PCR (RT-QPCR)

Total RNA (1 g) was reverse-transcribed with random primers using the Promega AMV Reverse Transcriptase kit (Promega, Madison, Wis.) according to the recommendations of the manufacturer. PCR amplifications were performed using the ABI PRISM 7700 Sequence Detection System (Perkin Elmer Applied Biosystems, Foster, Calif.). Reactions were performed in a 50 μl reaction mixture that included 25 μl SYBR Green JumpStart Taq ReadyMix (Sigma, St Louis, Mo.), distilled H₂O, DNA template, and 0.2 μM each primer from QuantiteTect primer assays (Qiagen, Valencia, Calif.). Amplification was performed by initial denaturation at 94° C. for 2 min, and 40 cycles of denaturation at 95° C. for 15 s, annealing at 60° C. for 1 min, and extension for 1 min at 72° C. This was followed by a dissociation cycle of 95° C. for 15 s, 60° C. for 15 s, and 95° C. for 15 s. Threshold cycles (C_{T}) for both Fra-1 mRNAs and the 18S rRNA control were determined. Original input RNA amounts were calculated using the Comparative C_{T} Method (2^{-dd̂C}T) to analyze changes in gene expression in the samples relative to the untreated control sample. Duplicate assays were performed with RNA samples isolated from at least 2 independent experiments. The values obtained from cDNAs and 18S controls provided relative gene expression levels for the gene loci investigated [24].

### Immunofluorescence

Dual confocal fluorescence approaches were used to determine if co-localization of Fra-1 was specific to nuclear PCNA-positive cells. Cells, grown on coverslips, were fixed in 100% methanol for 1 h on ice, washed in PBS, and incubated in 0.1% Tween-20 in PBS for 30 min at room temperature (RT). After incubation in blocking solution (2% dry milk, 0.1% Tween-20 in PBS) for 30 min at RT, cells were incubated with a cocktail of primary antibodies [mouse anti-PCNA (Pharmagen; 1:1,000) and rabbit polyclonal anti-Fra-1 antibody (R-20) (Santa Cruz Biotechnology Inc., 1:100) for 1 h at RT. Cells were washed twice for 20 min in blocking solution, and once for 10 min in PBS. PCNA was detected using Alexa Fluor 647-goat-anti-mouse IgG (Molecular Probes), diluted 1:400 in 10 μg/ml BSA/PBS. Fra-1 was detected using Alexa Fluor 568-goat-anti-rabbit IgG (Molecular Probes), diluted 1:200 in 10 μg/ml BSA/PBS. Controls were run using only primary or secondary antibodies. Following a final wash in PBS, sections were counter-stained with SYTOX green (1:1,000 in PBS) (Molecular Probes, Eugene, Oreg.), washed 1× in PBS, and mounted on glass slides using AquaPoly/Mount (Polysciences Inc., Warrington, Pa.), and examined using confocal scanning laser microscopy. For each sample, confocal images were collected in fluorescence modes, followed by electronic merging of the images.

### Statistical Analyses

All experiments used multiple replicate determinations (N=2, 3 or 8) per group, per time point. Experiments were performed in duplicate. Results were evaluated by one-way analysis of variance using the Student-Newman-Keuls procedure for adjustment of multiple pairwise comparisons between treatment groups. Differences with P values ≦0.05 were considered statistically significant.

### Results

### The Combination of Ranpirnase and Rosiglitazone Synergistically Reduced Viability and Increased Death in Several Cancer Cell Lines.

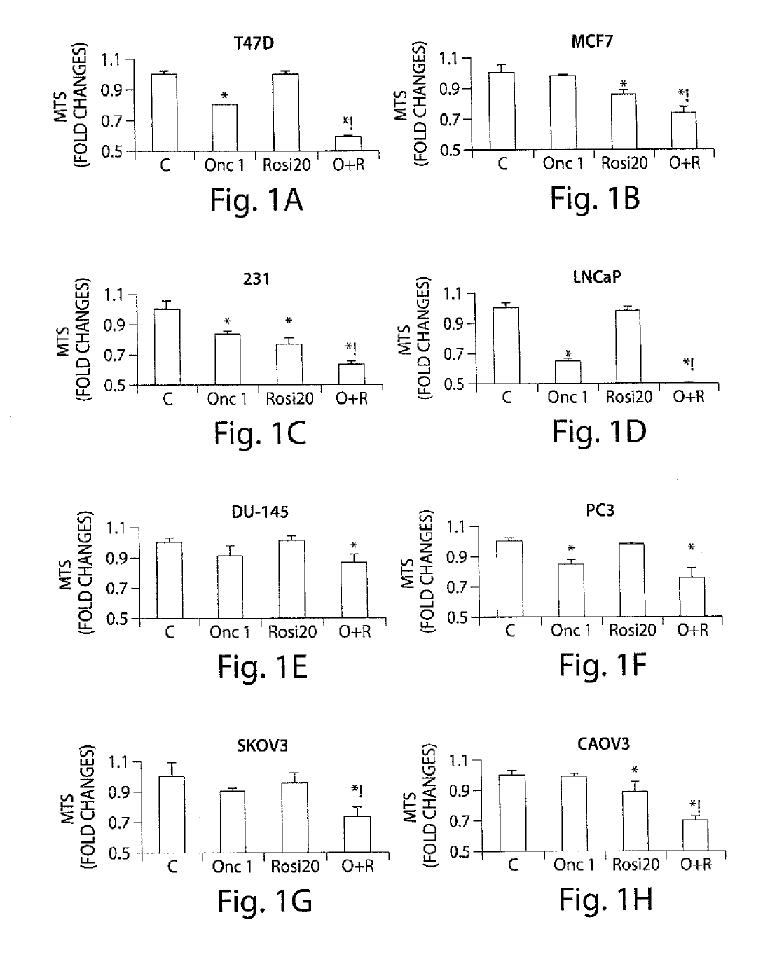

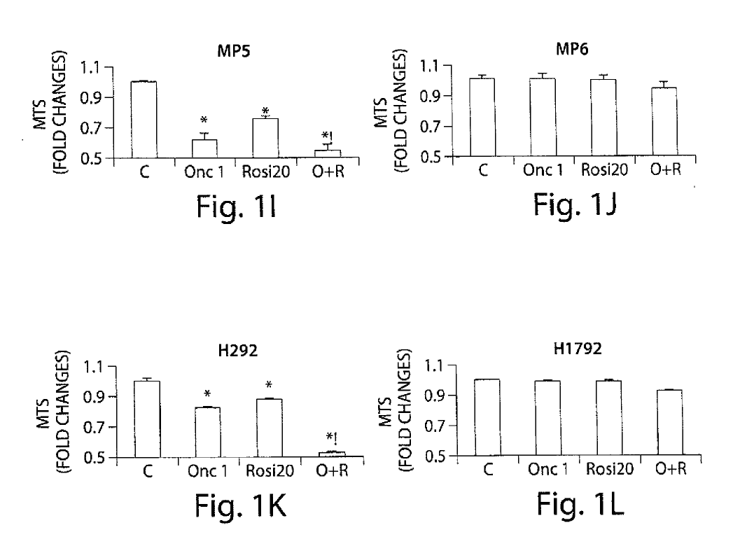

To test the hypothesis that the combination of the two drugs is synergistically anti-neoplastic, all 12 cancer cell lines were treated with ranpirnase (1 μg/mL), rosiglitazone (20 M) or their combination for 48 h. Cell viability measured by MTS assay showed (FIG. 1) that the combination of ranpirnase and rosiglitazone had a significant synergistic effect on the reduction of cell viability in seven out of the twelve cell lines tested (FIG. 1).

### The Combination of Ranpirnase and Rosiglitazone Increased the Proportions of Hypodiploid (SubG₀/G₁) Cells.

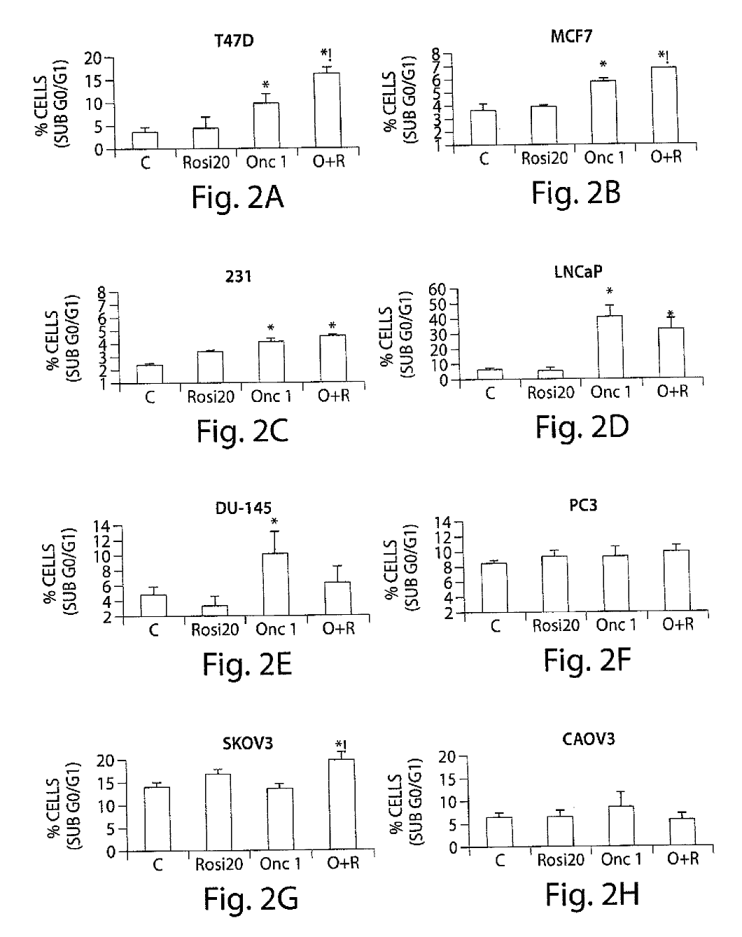

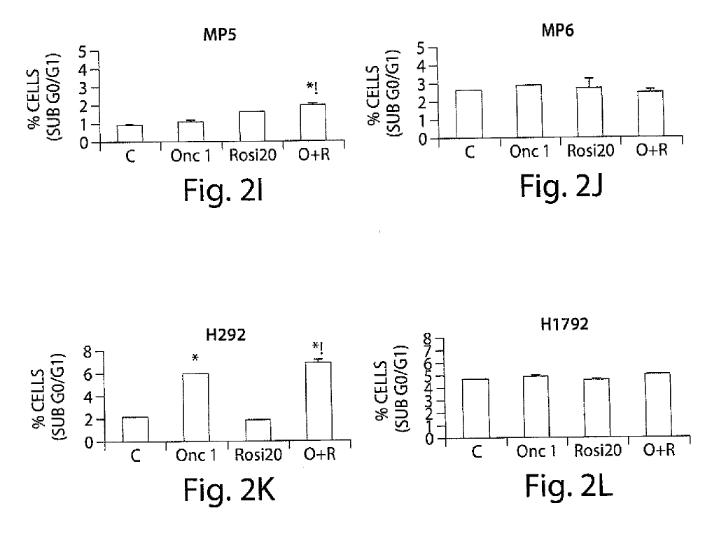

To prove the hypothesis that ranpirnase increased cell killing cooperatively with rosiglitazone, the effects of ranpirnase (1 μg/ml), rosiglitazone (20 μM), and their combination, were determined on the cell cycle kinetics of the 12 cancer cell lines. As early as 48 hours after treatment, five of the twelve cell lines showed a significant increases (P≦0.05) in the proportions of cells in subG₀/G₁ (apoptotic and necrotic cells) (FIG. 2). These changes occurred with concomitant decreases in cells in G₀/G₁ and increases in the numbers of cells in G₂/M, suggesting cell cycle arrest.

### The Combination of Ranpirnase and Rosiglitazone Triggered a Reduction in Proliferation and Increased Apoptosis in Several Cancer Cell Lines.

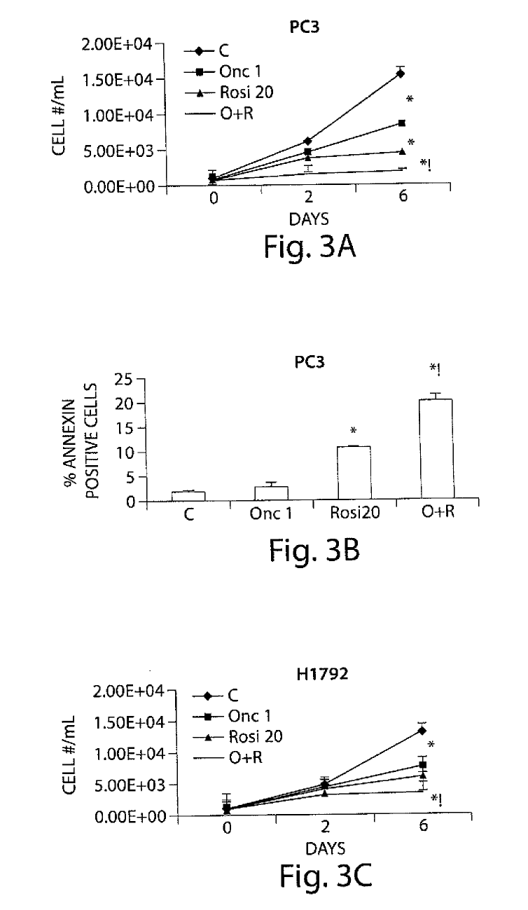

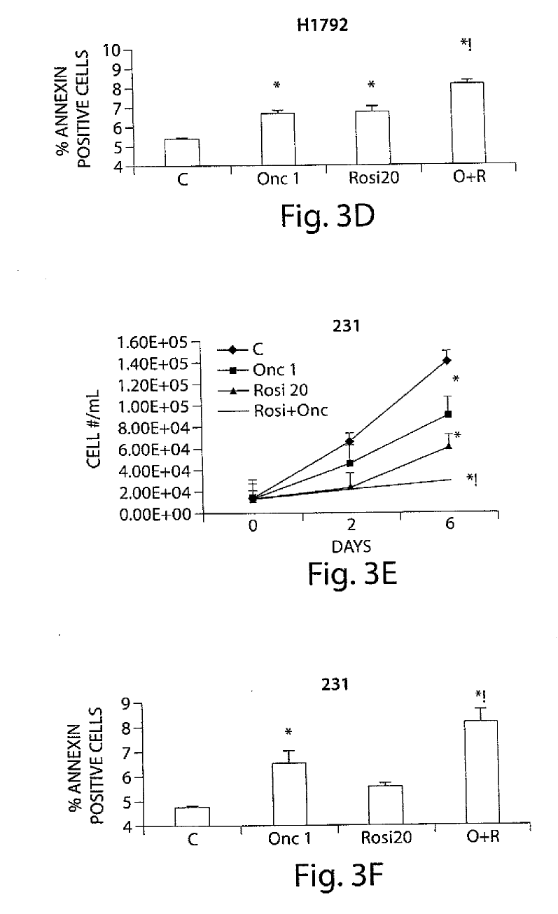

To determine the mechanism of cell death from the combination of ranpirnase and rosiglitazone, PC3, H1792 and 231 cell lines were examined using the Annexin V assay to detect apoptotic cell death after 6 days of treatment (FIG. 3B, D, and F). These cell lines were originally selected because of their resistance to the drug combination after 48 hours of treatment in previous tests (FIG. 2). Compared to control cells, significantly higher concentrations of Annexin V-positive cells were observed. Longer term treatment showed that even these relatively resistant cell lines underwent apoptosis when exposed to the combination for 6 days. Growth curves (FIG. 3A, C and E) also showed a synergistic decrease in cell growth at six days with the combination of ranpirnase and rosiglitazone.

### The Combination of Ranpirnase and Rosiglitazone Decreased Expression of Fra-1 and Survivin.

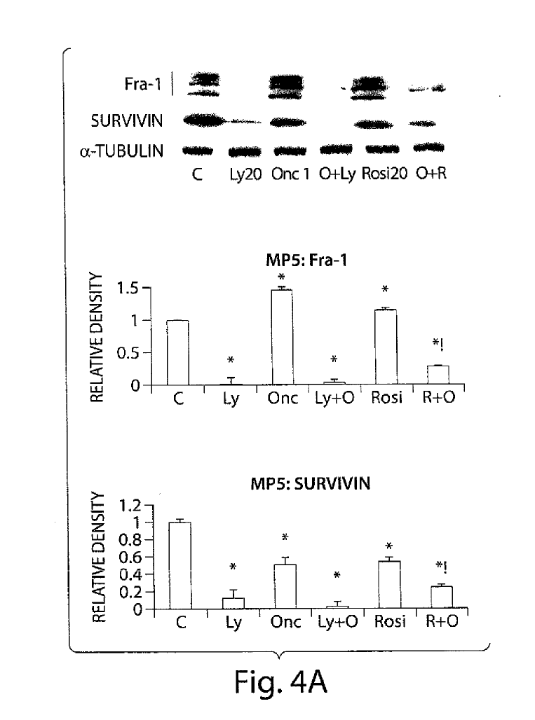

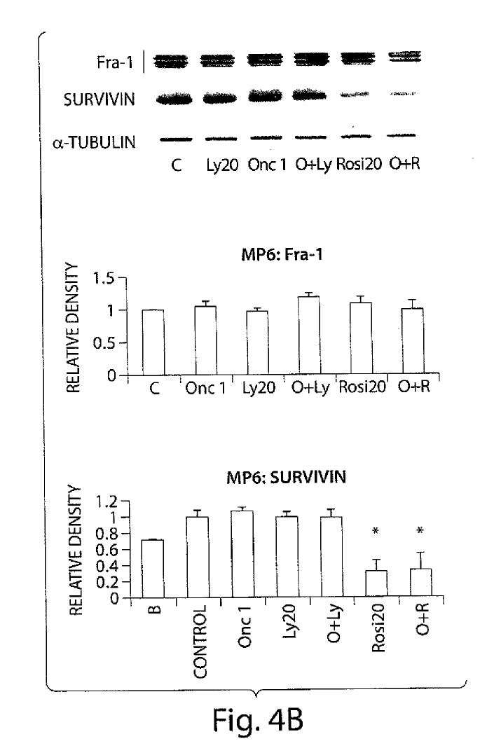

Fra-1 expression in mesothelioma cell lines is regulated by the ERK1/2 or PI3K pathways in a cell-dependent manner [24]. To determine which of these two pathways is affected by the combination, two mesothelioma cell lines were selected for testing. The MP5 line has been previously shown to have a PI3K-dependent Fra-1 expression, while the MP6 line has been shown to have an ERK1/2-dependent Fra-1 expression [24]. Ranpirnase and rosiglitazone in combination down-regulated Fra-1 only in the cell line with a PI3K-dependent Fra-1 expression, but not in the other cell line (FIG. 4). The use of the PI3K inhibitor LY294002 further confirmed the PI3K effect on Fra-1 expression.

Survivin, another PI3K-dependent protein, was also down-regulated by the PI3K inhibitor, and by the combination of ranpirnase and rosiglitazone in the MP5 cell line, but only by the drug combination in the MP6 cell line.

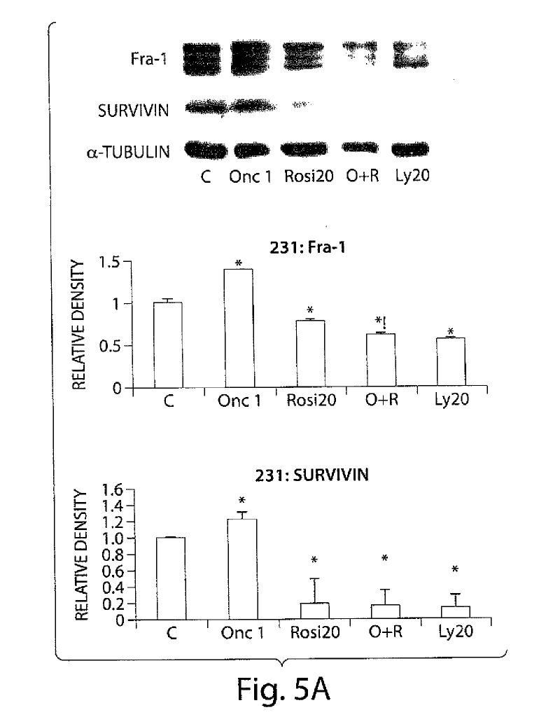

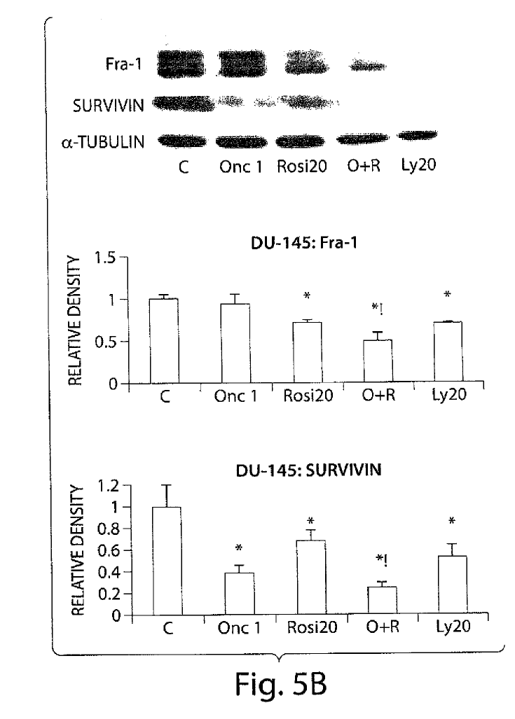

A second set of cancer lines (231 and DU-145) was treated for 48 h with ranpirnase, rosiglitazone alone, or in combination, and then washed and incubated for three hours with fresh DMEM/F12 complete media containing 0.5% FBS and 1 μM insulin. These conditions were previously shown to result in a peak Fra-1 expression in these cells lines. Results under these favorable conditions for Fra-1 expression still showed a synergistic down-regulation of Fra-1 in both cell lines (FIG. 5). Down-regulation of Survivin was observed only in the DU-145 cell line.

### Decreased Expression of Fra-1 Induced Apoptosis.

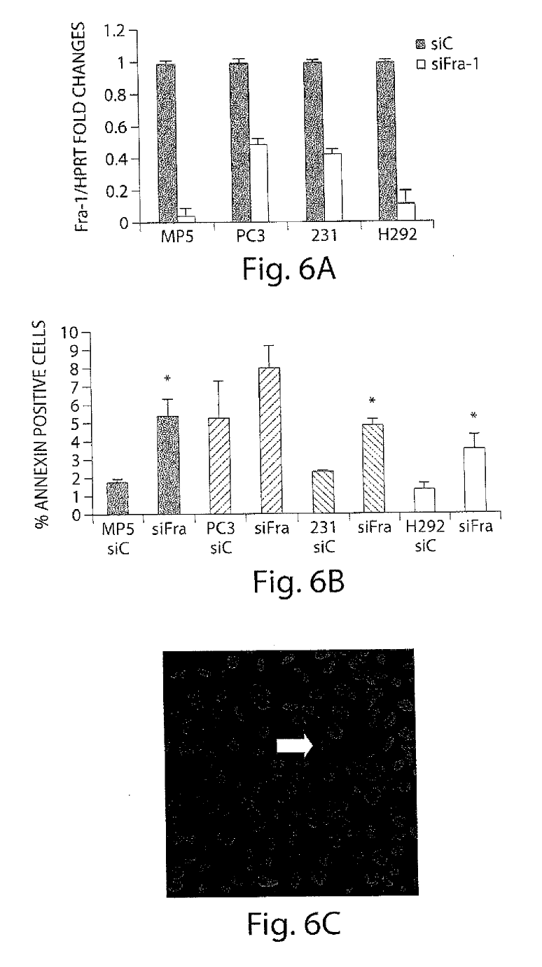

To determine if the down-regulation of Fra-1 accounted for the apoptotic effect produced by the drug combination, all cell lines were transfected with siFra-1 or scramble control and tested for Fra-1 knock-out by RT-QPCR. Only the cells demonstrating >50% reduction in Fra-1 expression were used in this experiment (FIG. 6A). After cell transfection with the RNAi, cells were left for 5 days and tested for apoptosis using the Annexin V assay. As shown in FIG. 6B, significantly increased apoptosis was observed in cell lines MP5, 231 and H292. The use of immunofluorescence showed that proliferating cells expressed nuclear Fra-1; dying cells did not (FIG. 6B).

### Fra-1 Expression Increased Drug Resistance to Ranpirnase

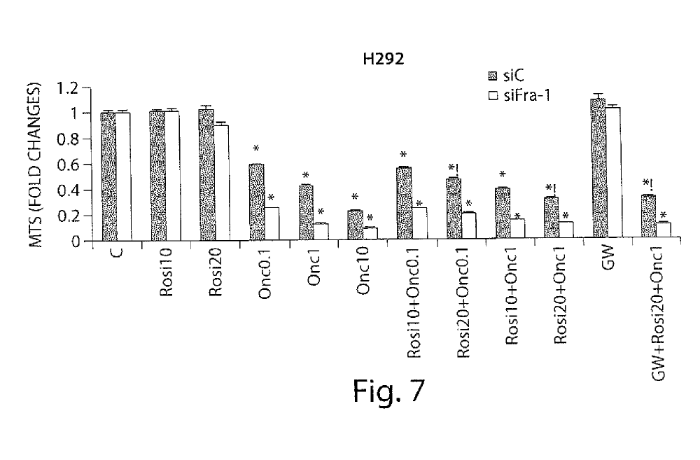

To directly observe if the knock-down of Fra-1 increased the efficacy of ranpirnase, rosiglitazone, or their combination, cell line H292 (a cell line with high transfection efficiency) was studied. Cells were transfected with siFra-1 or scramble control (siC) and left to rest for 48 h before treatment. Cells were then treated with different concentrations of ranpirnase (0.1, 1 or 10 μg/mL), rosiglitazone (10 or 20 μM), or their combination for 48 h. The PPARγ inhibitor GW9662 (2 μM) was added to observe the influence of PPARγ. FIG. 7 shows that ranpirnase reduced viability in a dose-dependent manner. The knock-down of Fra-1 significantly increased the efficacy of ranpirnase alone at all concentrations, but not rosiglitazone alone. The use of the drug combination produced a synergistic effect on the scramble control (siC) transfected cells, but not in Fra-1 knock-down cells. The synergistic effect of rosiglitazone and ranpirnase was not changed by the modulation of PPARγ.

### Discussion

In human mesothelioma cell lines, it has previously been shown that the increased expression of survival pathways, frequently activated in cancer as the PI3K pathway [26], predicts the efficacy of chemotherapeutic drugs such as cisplatin and ranpirnase [19, 27]. The data presented here demonstrate that the combination of ranpirnase with rosiglitazone, an anti-diabetic drug targeting the PI3K pathway [21], results in synergistic killing of several cancer cell lines. The mechanism of cell death was determined to involve cell apoptosis. It was also investigated whether two recognized PI3K-regulated proteins (Fra-1 [24] and Survivin [25]), key to the development and maintenance of several cancers, were modulated by the drug combination. Furthermore, the effect of knocking down Fra-1 on the cell killing and apoptosis found with combinations of ranpirnase with rosiglitazone, was also investigated.

Under the experimental conditions of the studies, the combination of ranpirnase and rosiglitazone down-regulated Fra-1 in a cell dependent manner. It has previously been shown that the regulation of Fra-1 in mesothelioma could be dependent on the ERK1/ERK2 pathway [24, 28], or on the PI3K pathway [24]. The down-regulating effect on Fra-1 produced by the combination of ranpirnase and rosiglitazone suggest that the combination may act through PI3K.

Results presented here also show that the knock-down of Fra-1 could induce apoptosis. This finding could partially explain the killing effect of the drug combination in these cell lines. The knock-down of Fra-1 in the cell line H292 further demonstrates that the synergistic effect of the two drugs is partially related to the modulation of Fra-1. To test whether the synergism between these two drugs was related to rosiglitazone's ability to activate PPARγ, a PPARγ antagonist was used together with the combination. The synergistic effect was independent of PPARγ.

Clinical trials of ranpirnase alone (Onconase™) showed heterogeneity in therapeutic efficacy [19]. These differential responses might reflect varying survival signaling mechanisms. The results presented herein suggest that combined therapeutic use of ranpirnase and rosiglitazone may overcome the resistance produced in some cancer cells by the activation of survival pathways and their targets.

### REFERENCES FOR EXAMPLE 1

1. Hu Y C, Lam K Y, Law S, Wong J, Srivastava G: Clin Cancer Res 2001, 7:2213-2221.
2. Chiappetta G, Ferraro A, Botti G, Monaco M, Pasquinelli R, Vuttariello E, Arnaldi L, Di Bonito M, D'Aiuto G, Pierantoni G M, Fusco A: BMC Cancer 2007, 7:17.
3. Zajchowski D, Bartholdi M, Gong Y, Webster L, Liu H, Munishkin A, Beauheim C, Harvey S, Ethier S, Johnson P: Cancer Res 2001, 61:5168-5178.
4. Kustikova O, Kramerov D, Grigorian M, Berezin V, Bock E, Lukanidin E, Tulchinsky E: Mol Cell Biol 1998, 18:7095-7105.
5. Mechta F, Lallemand D, Pfarr C M, Yaniv M: Oncogene 1997, 14:837-847.
6. Jochum W, David J P, Elliott C, Wutz A, Plenk H, Jr., Matsuo K, Wagner E F: Nat Med 2000, 6:980-984.
7. Reddy S P, Vuong H, Adiseshaiah P: Am J Respir Crit Care Med 2002, 165:A828.
8. Ramos-Nino M E, Timblin C R, Mossman B T: Cancer Res 2002, 62:6065-6069.
9. Adida C, Crotty P L, McGrath J, Berrebi D, Diebold J, Altieri D C: Am J Pathol 1998, 152:43-49.
10. Ambrosini G, Adida C, Altieri D C: Nat Med 1997, 3:917-921.
11. Johnson M E, Howerth E W: Vet Pathol 2004, 41:599-607.
12. Ramos-Nino M: Drugs of the Future 2007, 32:517-526.
13. Halicka H D, Murakami T, Papageorgio C N, Mittelman A, Mikulski S M, Shogen K, Darzynkiewicz Z: Cell Prolif 2000, 33:407-417.
14. Lee I, Lee Y H, Mikulski S M, Lee J, Covone K, Shogen K: J Surg Oncol 2000, 73:164-171.
15. Darzynkiewicz Z, Carter M, Mikulski S, Ardelt W, Shogen K: Cell Tissue Kinet 1988, 21:169-182.
16. Rybak S M, Pearson J W, Fogler W E, Volker K, Spence S E, Newton D L, Mikulski S M, Ardelt W, Riggs C W, Kung H F, Longo D L: J Natl Cancer Inst 1996, 88:747-753.
17. Juan G, Ardelt B, Li X, Mikulski S, Shogen K, Ardelt W, Mittelman A, Darzynkiewicz Z: Leukemia 1998, 8:1241-1248.
18. Newton D L, Hansen H J, Mikulski S M, Goldenberg D M, Rybak S M: Blood 2001, 97:528-535.
19. Ramos-Nino M E, Vianale G, Sabo-Attwood T, Mutti L, Porta C, Heintz N, Mossman B T: Mol Cancer Ther 2005, 4:835-842.
20. Mikulski S M, Ardelt W, Shogen K, Bernstein E H, Menduke H: J Natl Cancer Inst 1990, 82:151-153.
21. Han S, Roman J: Mol Cancer Ther 2006, 5:430-437.
22. Nunez M, Martin G, Cocca C, Mohamad N, Gutierrez A, Cricco G, Medina V, Rivera E, Croci M, Crescenti E, Bergoc R: Anticancer Res 2006, 26:2113-2122.
23. Russu W A: Med Hypotheses 2007, 68:343-346.
24. Ramos-Nino M E, Blumen S R, Sabo-Attwood T, Pass H, Carbone M, Testa J, Altomare D A, Mossman B T: Am J Respir Cell Mol Biol 2007.
25. Kawamura K, Fukuda J, Shimizu Y, Kodama H, Tanaka T: Biol Reprod 2005, 73:1094-1101.
26. Vivanco I, Sawyers C L: Nat Rev Cancer 2002, 2:489-501.
27. Altomare D A, You H, Xiao G H, Ramos-Nino M E, Skele K L, De Rienzo A, Jhanwar S C, Mossman B T, Kane A B, Testa J R: Oncogene 2005, 24:6080-6089.
28. Ramos-Nino M, Timblin C, Mossman B: Cancer Res 2002, 62:6065-6069.
29. BeruBe K, Quinlan T, Fung H, Magae J, Vacek P, Taatjes D, Mossman B: Am J Respir Cell Mol Biol 1996, 15:141-147.
30. Scapoli L, Ramos-Nino M E, Martinelli M, Mossman B T: Oncogene 2004, 23:805-813.

### Example 2

### Investigation into Rate of Tumor Growth Following Treatment with an RNase and Thiazolidinedione Compound in a Xenograft Tumor Model

The organ-specific environment is an important factor for the growth and progression of tumors in vivo. Xenograft tumor models have been very useful to test for tumorigenicity. Data presented in Example 1 showed the efficacy of the combination of the RNAse, Onconase™ and the thiazolidinedione, Rosiglitazone in down-regulating the expression of Fra-1 in vitro. Experiments presented in Example 2 test whether this efficacy can be maintained in vivo. Here, the effect of Onconase™, and Rosiglitazone alone or in combination is determined in tumor growth, induction of apoptosis, and expression of Fra-1 and Fra-1 target genes using a xenograft tumor model.

### Materials and Methods

The cell lines DU-145 and MDA-MB-231 are used, as representatives of cell lines with high basal Fra-1 expression. Cells are inoculated into BALB/c nu/nu mice (N=14) per treatment as determined by power calculation (80% power to detect a 25% tumor reduction with an alpha=0.05). Cancer cells are left to establish themselves until about 8 mm in diameter. Then, they are randomly assigned to control or treatment groups. The growth and weight of tumors is monitored for 8 weeks to determine the percentage of tumor growth inhibition. Tumors are fixed for histology and immunohistochemistry to determine apoptosis and expression of Fra-1, and Fra-1 target genes.

### Compounds

Onconase™ (trademark: Onconase® and generic name: ranpirnase) is supplied by the Alfacell Corporation (Bloomfield, N.J., USA). Original stock solutions of Onconase™ at 5 mg/ml is made in sterile distilled water and frozen at −20° C. until needed. Prior to experiments, Onconase™ is thawed and diluted to the appropriate concentrations in saline for in vivo work. The thiazolidinedione, Rosiglitazone (Sigma-Aldrich) stock solutions are diluted in DMSO. Prior to experiments Rosiglitazone is made to the appropriate concentration with 0.5% carboxymethylcellulose to obtain a maximum final DMSO concentration of 10% for in vivo work.

### Chemotherapeutic Experiments

BALB/c nude mice are weighed (20-22 g) at the start of the experiment. The mice are housed in sterilized filter-topped cages and maintained in sterile conditions. The two human tumor cell lines are cultured as indicated previously. On the day of implantation, cells are harvested by incubation with trypsin, washed and diluted in cultured media. Cell viability is determined by trypan blue dye exclusion to obtain a suspension with >95% viability. Animals are anesthetized with 2% Rompun (Bayer Pharma) at 5 mg/Kg and Zoletil 100 (Virbac) at 30 mg/Kg, administered i.p. Tumor cells are implanted subcutaneously in single cell suspension of 100 μl (1-2×10⁶ cells per ml) using a 26-gauge needle at two positions into the back of 2-month-old BALB/c (nu/nu) mice. Each cell line is assayed into 14 control and 14 treatment animals. Animals are offered food and water ad libitum during the treatment period. Tumor growth and animal weight are recorded daily for 6 weeks after injection or when it reaches more than 20% of the body weight. Following euthanasia of mice with sodium pentobarbital (i.p.), tumor volumes are calculated using the formula V=0.4×AB², with A and B as the longer and shorter diameters of the tumor (Lee et al., J Surg Oncol 2000, 73(3):164-71). Tumors are then placed in 4% paraformaldehyde O/N and sent to the pathology core facility to be immersed in paraffin blocks for histology and immunohistochemistry. A second tumor is divided in two sections, minced, and placed in RNA later solution (Ambion, Austin, Tex.) for isolation of RNA or freeze frozen for protein analysis. All animal protocols are approved by the Animal Care and Use Committee, University of Vermont, Burlington, Vt. The number of animals used for this aim are: 56 (28 per cell line) per treatment (Control, Rosiglitazone low dose alone, Rosiglitazone high dose alone, Onconase™ alone, Rosiglitazone low dose plus Onconase™ or Rosiglitazone high dose plus Onconase™) for a total of 336 animals.

### Chemotherapeutic Treatment Schedules

Animals with established tumors of 8-9 mm diameter are randomly assigned to treatment or vehicle groups. Animals assigned to the treatment groups receive one of the following treatments: 1) i.v. injection of Onconase™ at 2.5 mg/kg, twice a week (on Monday and Thursday) for 2 weeks at a volume of 0.1 ml/20 g of body weight. This dose was found in previous studies (Dr Shogen-Alphacell personal communication) to be effective in producing apoptosis, but non-toxic to the animals. 2) one of two doses of Rosiglitazone (a low dose of 20 mg/Kg/d; and a high dose of 150 mg/Kg/d) in 10% DMSO and 0.5% carboxymethylcellulose administer by oral gavage starting when the tumors reach 8 mm in diameter for the duration of the experiment as described previously (Heaney et al., J Clin Invest 2003, 111(9):1381-8) a combination of Onconase™ with a low or high dose of Rosiglitazone. Animal mortality is checked daily, and the antitumor activity is evaluated as follows: Tumor reduction %=(Mean volume size of treated group/mean tumor size of control group)×100, 60 days after injection of tumor cells or when tumor burden is no more than 20% of the animal weight. This protocol is adjusted after a pilot test is completed. All experiments are done at the final protocol.

### Histological Study

Tumors are removed and fixed in 4% phosphate-buffered formalin overnight and embedded in paraffin. Sections of 4 um are stained with H&E for microscopic evaluation and send to Pathology Core Facility for analysis.

### Immunohistochemistry

Slides from paraffin sections are prepared and processed for TUNEL staining as described before (Ramos-Nino et al., Mol Cancer Ther 2005 4(5):835-42) for determining the status of apoptosis. At least 200 cells are counted from each experiment, with data reported as the percentage of apoptotic cells. Tumor sections are deparaffinized in xylene (2×15 min) and rehydrated in a graded ethanol series (95% to 50%) and then rinsed in water. A TUNEL assay for detection of DNA strand breaks is performed using a commercial kit following the manufacturer's instructions (Promega Corporation, Madison, Wis.). Slides are washed with fresh PBS several times, and cells are permeabilized using 0.2% Triton X-100 solution in PBS. After cell incubation with 100 μl of equilibration buffer, a biotinylated nucleotide mix and TdT reaction mix is added at 37° C. for 1 hour in a humidified chamber. The reaction terminates with 2×SSC for 15 minutes. Endogenous peroxidases are blocked by immersing the slides in 0.3% H₂O₂ for 5 min. Slides are then incubated with Streptavidin HRP complex for 30 minutes, stained with 3-3′ diaminobenzidine tetrahydrochloride, and counterstained with hematoxylin to detect the apoptotic/necrotic nuclei. Negative controls include cells incubated in enzyme, and positive controls consist of cells treated with DNAse I (Promega Corporation, Madison, Wis.) (Ramos-Nino et al., Mol Cancer Ther 2005 4(5):835-42).

### RNA Extraction

Tumor tissue is stored in RNA later solution following the manufacturer's protocol. To isolate RNA, approximately 40-80 mg of tumor tissue is homogenized in Trizol and extracted with chloroform. The RNA is precipitated with isopropanol, washed with 75% ethanol, and further purified using Qiagen's RNeasy system (Sabo-Attwood et al., Am J Pathol 2005, 167(5):1243-56). The isolated RNA is further treated with DNAse I (Ambion, Austin, Tex.). Quantitation follows by measuring the absorbance at 260 nm, and quality assessed on an Agilent 2100 bioanalyzer (Palo Alto, Calif.). Total RNA (1 μg) is reverse-transcribed with random primers using the Promega AMV Reverse Transcriptase kit, according to recommendations of the manufacturer and RT-QRTPCR (as described in the previous section) reactions are performed to determined levels of Fra-1 expression and Fra-1 target genes. Also the expression of PPAR-gamma (an important target of Rosiglitazone is evaluated). All primers sets for RT-QRTPCR for potential genes: Fra-1, Dicer, CD44, c-Met, Angiopoietin-like 4, Glut1/3 have been acquired (Qiagen Syber-Green based pre-design sets) and tested.

### Protein Profiling

For protein profiling, whole cell protein lysates from treated and control tumors are isolated following a previously described protocol (Ramos-Nino et al., Am J Respir Cell Mol Biol, 2003 29(3 Suppl):S51-8). Briefly, tissue previously washed in cold-PBS is suspended and homogenized in 1 ml of lysis buffer per 250 mg wet weight of the chopped tissue (20 mM MOPS, pH 7.0; 2 mM EGTA; 5 mM EDTA; 30 mM NaF; 40 mM beta-glycerophosphate, pH 7.2; 10 mM sodium pyrophosphate; 2 mM sodium orthovanadate; 1 mM phenylmethylsulfonylfluoride; 3 mM benzamidine; 5 uM pepstatin A; 10 uM leupeptin and 0.5% Nonidet P-40, pH 7.0). Lysates are then sonicated 2×15 seconds, and the homogenate ultracentrifuged for 30 min at 100,000×g. Protein concentrations from the resulting supernatant fraction are measured using the Bradford assay (Bio Rad, Hercules, Calif.). 500 μg of protein per sample is suspended in SDS-PAGE sample buffer as specified by Laemmli. Protein samples are shipped to Kinexus on dry ice, processed using the Kinetworks phospho-site broad coverage pathway 11.0 (Kinexus), and immunoreactive proteins are quantified with a high resolution scanner that detects chemiluminescence. This profile tracks 37 phosphorylation sites in phosphoproteins with antibodies that recognize phosphorylated epitopes. The status of activation of kinases in the ERK, PI3K/AKT and WNT/beta-Catenin pathway important in Fra-1 regulation are among the kinases tested in these screens. Data is presented as fold change in protein expression or modification with respect to untreated control samples. Only those signaling kinases exhibiting fold changes >1.5 are considered altered in expression and graphed. Western Blots are employed to validate at least 5 changes in protein levels or activation states observed by protein profiling assays. All antibodies are purchased from commercial sources. Whole cell lysates are separated by SDS-PAGE electrophoresis and transferred to a nitrocellulose membrane as previously described. Membranes are blocked in 5% Blotto prior to addition of primary antibody. After 16 hours, the membrane is washed with Tris-buffered saline with Tween (TBST) and incubated with the appropriate secondary antibody complexed with HRP for 1 hour. After washing, bound antibody is detected using ECL kits (Amersham/GE Healthcare) following the manufactures instruction. All membranes are stripped and reprobed with the appropriate loading control and all blots are semi-quantified using Quantity One software (Bio-Rad).

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

### EQUIVALENTS

Those skilled in the art will recognize, or be able to ascertain using no more than routine experimentation, many equivalents to the specific embodiments of the invention described herein. Such equivalents are intended to be encompassed by the following claims.

All references, including patent documents, disclosed herein are incorporated by reference in their entirety.

What is claimed is:

1. A method for treating a cancer in a subject, the method comprising:
co-administering to a subject in need of such treatment, an amount of a ribonuclease compound and an amount of a thiazolidinedione compound, wherein the compounds are co-administered in amounts therapeutically effective to treat the cancer in the subject.

2. The method of claim 1, wherein the ribonuclease compound is ranpirnase.

3. The method of claim 1, wherein the thiazolidinedione compound is rosiglitazone.

4. The method of claim 1, wherein the cancer is an adenocarcinoma.

5. The method of claim 4, wherein the adenocarcinoma is lung cancer.

6. The method of claim 4, wherein the adenocarcinoma is breast cancer.

7. The method of claim 4, wherein the adenocarcinoma is prostate cancer.

8. The method of claim 1, wherein the cancer is mesothelioma.

9. The method of claim 1, wherein the cancer is a solid tumor.

10. The method of claim 1, wherein the cancer exhibits PI3K-dependent Fra-1 expression.

11. The method of claim 1, wherein the cancer exhibits high survivin expression.

12. The method of claim 1, wherein the subject is a mammal.

13. The method of claim 12, wherein the subject is a human.

14. The method of claim 1, wherein the ribonuclease compound and the thiazolidinedione compound are co-administered in one or more pharmaceutical compositions.

15. The method of claim 14, wherein the one or more pharmaceutical compositions further comprise at least one pharmaceutically acceptable carrier, diluent, excipient, or adjuvant.

16. The method of claim 1, further comprising administering an additional chemotherapeutic agent to the subject.

17. The method of claim 1, wherein the subject has cancer.

18. The method of claim 1, wherein the subject is suspected of having cancer.

19. The method of claim 1, wherein co-administration comprises administering the ribonuclease compound at least every 1 day, 2 days, 3 days, 4 days, 5 days, 6 days, 1 week, 2 weeks, 3 weeks, 4 weeks, 5 weeks, 6 weeks, 7 weeks, 8 weeks, 2 months, 3 months, 4 months, 5 months, 6 months, 7 months, 8 months, 9 months, 10 months, 11 months, or 12 months.

20. The method of claim 1, wherein co-administration comprises administering the thiazolidinedione compound at least every 1 day, 2 days, 3 days, 4 days, 5 days, 6 days, 1 week, 2 weeks, 3 weeks, 4 weeks, 5 weeks, 6 weeks, 7 weeks, 8 weeks, 2 months, 3 months, 4 months, 5 months, 6 months, 7 months, 8 months, 9 months, 10 months, 11, or 12 months.

21. The method of claim 1, wherein the co-administration of the ribonuclease compound and the thiazolidinedione compound to the subject is repeated one or more times.

22. A method for decreasing viability of a cancer cell, the method comprising:
contacting the cancer cell with a combination of a ribonuclease compound and a thiazolidinedione compound each in an amount effective such that the combination decreases the viability of the cancer cell.

23. The method of claim 22, wherein the ribonuclease compound is ranpirnase.

24. The method of claim 22, wherein the thiazolidinedione compound is rosiglitazone.

25. The method of claim 22, wherein the cell is contacted in vitro.

26. The method of claim 22, wherein the cell is contacted in vivo.

27. The method of claim 22, wherein the cell is a mammalian cell.

28. The method of claim 27, wherein the cell is a human cell.

29. The method of claim 22, wherein the cell is contacted simultaneously with the ribonuclease compound and the thiazolidinedione compound.

30. The method of claim 22, wherein the cancer is an adenocarcinoma.

31. The method of claim 30, wherein the adenocarcinoma is lung cancer.

32. The method of claim 30, wherein the adenocarcinoma is breast cancer.

33. The method of claim 30, wherein the adenocarcinoma is prostate cancer.

34. The method of claim 22, wherein the cancer is mesothelioma.

35. The method of claim 22, wherein the cancer is a solid tumor.

36. The method of claim 22, wherein the cancer exhibits PI3K-dependent Fra-1 expression.

37. The method of claim 22, wherein the cancer exhibits high survivin expression.

38. A method for assessing effectiveness of a combination of a ribonuclease compound and a thiazolidinedione compound for treatment of a cancer, the method comprising:
contacting a cell of the cancer with a combination of a ribonuclease compound and a thiazolidinedione compound; and
determining whether the contacted cell has reduced viability, wherein reduced viability indicates effectiveness of the combination of the ribonuclease compound and the thiazolidinedione compound for treatment of the cancer.

39. The method of claim 38, wherein the ribonuclease compound is ranpirnase.

40. The method of claim 38, wherein the thiazolidinedione compound is rosiglitazone.

41. The method of claim 38, wherein the cancer cell is contacted in vitro.

42. The method of claim 38, wherein the cancer cell is contacted in vivo.

43. The method of claim 38, wherein the cancer cell is a mammalian cancer cell.

44. The method of claim 43, wherein the mammalian cancer cell is a human cancer cell.

45. The method of claim 38, wherein the cancer cell is in contact with the ribonuclease compound at the same time the cancer cell is in contact with the thiazolidinedione compound.

46. The method of claim 38, wherein the cancer cell is from a subject.

47. The method of claim 46, further comprising selecting a combination ribonuclease compound and thiazolidinedione compound treatment regimen for the subject if it is determined that contact with the combination of the ribonuclease compound and the thiazolidinedione compound reduces viability of the cancer cell.

48. The method of claim 38, wherein the determination of the viability of the contacted cancer cell comprises comparing viability of the contacted cancer cell with viability of a control cancer cell.

49. The method of claim 48, wherein the control cancer cell is a cancer cell not contacted with the combination of the ribonuclease compound and the thiazolidinedione compound.

50. The method of claim 38, wherein the cancer is an adenocarcinoma.

51. The method of claim 50, wherein the adenocarcinoma is lung cancer.

52. The method of claim 50, wherein the adenocarcinoma is breast cancer.

53. The method of claim 50, wherein the adenocarcinoma is prostate cancer.

54. The method of claim 38, wherein the cancer is mesothelioma.

55. The method of claim 38, wherein the cancer is a solid tumor.

56. The method of claim 38, wherein the cancer exhibits PI3K-dependent Fra-1 expression.

57. The method of claim 38, wherein the cancer exhibits high survivin expression.

58. A kit for determining a treatment regimen for a cancer, the kit comprising:
a ribonuclease compound, a thiazolidinedione compound, and instructions for use of the two compounds for determining whether the combination of the two compounds can be used as a treatment regimen for the cancer.

59. The kit of claim 58, wherein a first container comprises the ribonuclease compound and a second container comprises the thiazolidinedione compound.

60. The kit of claim 58, wherein a first container comprises the ribonuclease compound and the thiazolidinedione compound.

61. The kit of claim 58, wherein the ribonuclease compound is ranpirnase.

62. The kit of claim 58, wherein the thiazolidinedione compound is rosiglitazone.

63. The kit of claim 58, wherein the cancer is an adenocarcinoma.

64. The kit of claim 63, wherein the adenocarcinoma is lung cancer.

65. The kit of claim 63, wherein the adenocarcinoma is breast cancer.

66. The kit of claim 63, wherein the adenocarcinoma is prostate cancer.

67. The kit of claim 58, wherein the cancer is mesothelioma.

68. The kit of claim 58, wherein the cancer is a solid tumor.

69. The kit of claim 58, wherein the cancer exhibits PI3K-dependent Fra-1 expression.

70. The kit of claim 58, wherein the cancer exhibits high survivin expression.

71. A kit for treating a cancer, the kit comprising:
a ribonuclease compound, a thiazolidinedione compound, and instructions for use of a combination of the compounds for treating the cancer.

72. The kit of claim 71, wherein a first container comprises the ribonuclease compound and a second container comprises the thiazolidinedione compound.

73. The kit of claim 71, wherein a first container comprises the ribonuclease compound and the thiazolidinedione compound.

74. The kit of claim 71, wherein the ribonuclease compound is ranpirnase.

75. The kit of claim 71, wherein the thiazolidinedione compound is rosiglitazone.

76. The kit of claim 71, wherein the cancer is an adenocarcinoma.

77. The kit of claim 76, wherein the adenocarcinoma is lung cancer.

78. The kit of claim 76, wherein the adenocarcinoma is breast cancer.

79. The kit of claim 76, wherein the adenocarcinoma is prostate cancer.

80. The kit of claim 71, wherein the cancer is mesothelioma.

81. The kit of claim 71, wherein the cancer is a solid tumor.

82. The kit of claim 71, wherein the cancer exhibits PI3K-dependent Fra-1 expression.

83. The kit of claim 71, wherein the cancer exhibits high survivin expression.
